SEQUENCE LISTING (10 sequences)

SEQ ID NO: 1 - DNA, 22 bp, Artificial Sequence
  Description of Artificial Sequence Synthetic      primer
gagaaatccaaagagcaaatgg

SEQ ID NO: 2 - DNA, 21 bp, Artificial Sequence
  Description of Artificial Sequence Synthetic      primer
agaatggaaaactggaatagg

SEQ ID NO: 3 - DNA, 20 bp, Artificial Sequence
  Description of Artificial Sequence Synthetic      primer
tcttcgatgcttcggattcc

SEQ ID NO: 4 - DNA, 21 bp, Artificial Sequence
  Description of Artificial Sequence Synthetic      primer
gaattctcggaatctctgttg

SEQ ID NO: 5 - DNA, 21 bp, Artificial Sequence
  Description of Artificial Sequence Synthetic      primer
ttacaagcagtgcagaaaacc

SEQ ID NO: 6 - DNA, 22 bp, Artificial Sequence
  Description of Artificial Sequence Synthetic      primer
agtaaacattgaaaccacagcc

SEQ ID NO: 7 - DNA, 20 bp, Artificial Sequence
  Description of Artificial Sequence Synthetic      primer
tctgcagctctgtgtgaagg

SEQ ID NO: 8 - DNA, 22 bp, Artificial Sequence
  Description of Artificial Sequence Synthetic      primer
cttcaaaaacttctccacaacc

SEQ ID NO: 9 - DNA, 17 bp, Artificial Sequence
  Description of Artificial Sequence Synthetic      primer
cccacgccacgctctcc

SEQ ID NO: 10 - DNA, 19 bp, Artificial Sequence
  Description of Artificial Sequence Synthetic      primer
tcctgtcagttggtgctcc

(19) United States
(12) Patent Application Publication
(10) Pub. No.: US 2019/0134100 A1
(43) Pub. Date: May 9, 2019

(54) METHOD OF INHIBITING ANGIOGENESIS

(71) Applicant: Janssen Biotech, Inc., Horsham, PA (US)

(72) Inventors: Jing Cao, Spring House, PA (US); Ian R. Harris, Spring House, PA (US)

(73) Assignee: Janssen Biotech, Inc., Horsham, PA (US)

(21) Appl. No.: 16/178,083

(22) Filed: Nov. 1, 2018

Related U.S. Application Data
(60) Provisional application No. 62/581,399, filed on Nov. 3, 2017.

(51) Int. Cl. A61K 35/51; A61P 27/02; C12N 5/0775

(52) U.S. Cl. CPC A61K 35/51; C12N 2501/165; C12N 5/0665; A61P 27/02

(57) ABSTRACT

Methods and compositions for treating ophthalmic disease and reducing retinal neovascularization using progenitor cells, such as postpartum-derived cells, and conditioned media produced from the cells, are disclosed.

### CROSS-REFERENCE TO RELATED APPLICATIONS

This application claims priority to U.S. Provisional Application No. 62/581,399, filed Nov. 3, 2017, the entire contents of which is incorporated by reference herein.

### FIELD OF THE INVENTION

This invention relates to the field of cell-based or regenerative therapy for ophthalmic diseases and disorders, particularly ocular conditions involving angiogenesis. The invention provides methods and compositions for inhibiting neovascularization and lowering of vascular endothelial growth factor (VEGF) using progenitor cells, such as umbilical cord-tissue derived cells, placenta tissue-derived cells, and conditioned media prepared from those cells.

### BACKGROUND OF THE INVENTION

Age-related macular degeneration (AMD) is the main cause of visual impairment and blindness in people aged over 65 years in developed countries. There are two forms of AMD, dry (atrophic) and wet (exudative). Wet AMD is characterized by the formation of choroidal neovascularization (CNV) which consequently leads to vision loss. About 85% of patients with AMD have the dry form that can turn into wet AMD at any time where ocular angiogenesis leads to irreversible visual impairment.

Vascular endothelial growth factor (VEGF) is the crucial regulator of angiogenesis and plays a critical role in the formation of neovascularization in wet AMD, leading to deterioration of central vision. Retinal pigment epithelium (RPE) cells are the major source of VEGF in wet AMD, contributing significantly to CNV formation. VEGF is overexpressed in the RPE of autopsy eyes with AMD and in RPE cells of surgically excised CNV membrane. Factors implicated in AMD, such as advanced glycation end products and reactive-oxygen intermediates, are potent stimuli of VEGF expression in RPE cells.

VEGF mediates its angiogenic effects by binding to specific VEGF receptors. Of the primary receptors, VEGFR1 and VEGFR2 are associated with angiogenesis, whereas VEGFR3 is associated with lymphangiogenesis. The soluble form of VEGFR1 results from alternative splicing of VEGFR1, and is a naturally occurring endogenous inhibitor of VEGF by sequestering VEGF from signaling receptors and forming non-signaling heterodimers with VEGFR2. Currently, anti-VEGF drugs are the standard care for this condition, but must be repeatedly administrated over a long period of time, burdening patients and often leading to undertreatment and subsequent vision loss.

Recently, efforts have been made to develop cell therapy for the treatment of retinal diseases, including AMD. AMD patients typically develop the dry form first; wet AMD occurs on a background of dry AMD. Therefore, dry AMD can sometimes be considered a precursor state for wet AMD. Geographic atrophy (GA) represents the greatest risk factor for advancing to wet AMD. Neovascular lesions are often present in the periphery of eyes with GA. The effect of hUTC on neovascularization in the eye is unclear.

### SUMMARY OF THE INVENTION

This invention provides compositions and methods applicable to cell-based or regenerative therapy for ophthalmic diseases and disorders. In particular, the invention features methods and compositions for treating ophthalmic disease or condition, including the regeneration or repair of ocular tissue using progenitor cells, such as postpartum-derived cells (PPDCs). The postpartum-derived cells may be umbilical cord tissue-derived cells (UTCs) or placental tissue-derived cells (PDCs).

One aspect of the invention is a method of inhibiting or reducing retinal neovascularization in retinopathy comprising administering a population of human umbilical cord tissue-derived cells to the eye of a subject with retinopathy. In embodiments, the human umbilical cord tissue-derived cells (hUTCs) are isolated from human umbilical cord tissue substantially free of blood. In embodiments, the human umbilical cord tissue-derived cells secrete VEGFR1. In embodiments, the VEGFR1 is human VEGFR1. In embodiments, the VEGFR1 is soluble VEGFR1.

Another embodiment includes a composition for use in inhibiting or reducing retinal neovascularization in retinopathy comprising a population of human umbilical cord tissue-derived cells. In embodiments, the human umbilical cord tissue-derived cells are isolated from human umbilical cord tissue substantially free of blood. Other embodiments relate to a population of postpartum-derived cells for use in inhibiting or reducing retinal neovascularization in retinopathy. In embodiments, the human umbilical cord tissue-derived cells secrete VEGFR1. In embodiments, the VEGFR1 is human VEGFR1. In embodiments, the VEGFR1 is soluble VEGFR1.

In the embodiments described herein, methods and compositions which use cells isolated from postpartum umbilical cord tissue may also use conditioned media produced from those cells. In the embodiments herein, the umbilical cord tissue-derived cells or conditioned media produced from those cells inhibit or reduce retinal neovascularization in retinopathy. In each of the embodiments described that use cells isolated from postpartum tissue, such as umbilical cord tissue, a composition comprising the cells may be used.

Another embodiment is a method of producing a conditioned media comprising human VEGFR1. The conditioned media is produced from human umbilical cord tissue-derived cells. In embodiments, the human umbilical cord tissue-derived cells are isolated from human umbilical cord tissue substantially free of blood. In embodiments, the human umbilical cord tissue-derived cells secrete VEGFR1. In embodiments, the VEGFR1 is human VEGFR1. In embodiments, the VEGFR1 is soluble VEGFR1.

A further embodiment is a method of inhibiting or reducing retinal neovascularization in retinopathy comprising administering a conditioned medium comprising VEGFR1. The conditioned media is produced from human umbilical cord tissue-derived cells. In embodiments, the human umbilical cord tissue-derived cells are isolated from human umbilical cord tissue substantially free of blood. In embodiments, the human umbilical cord tissue-derived cells secrete VEGFR1. In embodiments, the VEGFR1 is human VEGFR1.

One embodiment is a composition for use in reducing neoovascularization in retinopathy comprising administering a conditioned medium comprising VEGFR1. In embodiments, the VEGFR1 is human VEGFR1.

In the embodiments of the invention described herein, the postpartum-derived cells are derived from human umbilical cord tissue or placental tissue substantially free of blood. In embodiments, the cell is capable of expansion in culture and maintain a normal karyotype. The cell further comprises one or more of the following characteristics: (a) potential for at least about 40 doublings in culture; (b) attachment and expansion on a coated or uncoated tissue culture vessel, wherein the coated tissue culture vessel comprises a coating of gelatin, laminin, collagen, polyomithine, vitronectin, or fibronectin; (c) production of at least one of tissue factor, vimentin, or alpha-smooth muscle actin; (d) production of at least one of CD10, CD13, CD44, CD73, CD90, PDGFr-alpha, PD-L2 and HLA-A,B,C; (e) lack of production of at least one of CD31, CD34, CD45, CD80, CD86, CD117, CD141, CD178, B7-H2, HLA-G, and HLA-DR,DP,DQ, as detected by flow cytometry; (f) expression of a gene, which relative to a human cell that is a fibroblast, a mesenchymal stem cell, or an iliac crest bone marrow cell, is increased for at least one of a gene encoding: interleukin 8; reticulon 1; chemokine (C--X--C motif) ligand 1 (melonoma growth stimulating activity, alpha); chemokine (C--X--C motif) ligand 6 (granulocyte chemotactic protein 2); chemokine (C--X--C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113; (g) expression of a gene, which relative to a human cell that is a fibroblast, a mesenchymal stem cell, or an iliac crest bone marrow cell, is reduced for at least one of a gene encoding: short stature homeobox 2; heat shock 27 kDa protein 2; chemokine (C--X--C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (T AZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeo box 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; cytochrome c oxidase subunit VIIa polypeptide 1 (muscle); similar to neuralin 1; B cell translocation gene 1; hypothetical protein FLJ23191; and DKFZp586L151; and (h) lack expression of hTERT or telomerase. In one embodiment, the umbilical cord tissue-derived cell further has the characteristics of: (i) secretion of at least one of MCP-1, IL-6, IL-8, GCP-2, HGF, KGF, FGF, HB-EGF, BDNF, TPO, MIP1b, 1309, MDC, RANTES, and TIMP1; (j) lack of secretion of at least one of TGF-beta2, MIP1a, ANG2, PDGFbb, and VEGF, as detected by ELISA. In another embodiment, the placenta tissue-derived cell further has the characteristics of: (i) secretion of at least one of MCP-1, IL-6, IL-8, GCP-2, HGF, KGF, HB-EGF, BDNF, TPO, MIP1a, RANTES, and TIMP1; (j) lack of secretion of at least one of TGF-beta2, ANG2, PDGFbb, FGF, and VEGF, as detected by ELISA.

In specific embodiments as detailed herein, the postpartum-derived cell has all the identifying features of cell type UMB 022803 (P7) (ATCC Accession No. PTA-6067); cell type UMB 022803 (P17) (ATCC Accession No. PTA-6068), cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079). In an embodiment, the postpartum-derived cell derived from umbilicus tissue has all the identifying features of cell type UMB 022803 (P7) (ATCC Accession No. PTA-6067) or cell type UMB 022803 (P17) (ATCC Accession No. PTA-6068). In another embodiment, the postpartum-derived cell derived from placenta tissue has all the identifying features of cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079).

In embodiments as detailed herein, postpartum-derived cells are isolated in the presence of one or more enzyme activities comprising metalloprotease activity, mucolytic activity and neutral protease activity. Preferably, the cells have a normal karyotype, which is maintained as the cells are passaged in culture.

In embodiments, the postpartum derived cell population is isolated from human umbilical cord tissue substantially free of blood, is capable of expansion in culture, and expresses at least one of CD10, CD13, CD44, CD73, and CD90. In embodiments, the cell does not express CD31, CDE34, CD45 or CD117. In embodiments described herein, the postpartum derived cell is positive for HLA-A,B,C, and negative for HLA-DR,DP,DQ. In the embodiments described herein, the cells lack expression of hTERT or telomerase. In some embodiments, the cell expresses CD13, CD90 and HLA-ABC, and does not express CD31, CD34, CD45 and CD117.

In preferred embodiments, the postpartum-derived cells express each of CD10, CD13, CD44, CD73, and CD90. In some embodiments, the postpartum-derived cells express each of CD10, CD13, CD44, CD73, CD90, PDGFr-alpha, and HLA-A,B,C. In preferred embodiments, the postpartum-derived cells do not express any of CD31, CD34, CD45, CD117. In some embodiments, the postpartum-derived cells do not express any of CD31, CD34, CD45, CD117, CD141, or HLA-DR,DP,DQ, as detected by flow cytometry. In embodiments described above, the cell population is positive for HLA-A,B,C, and negative for HLA-DR,DP,DQ. In the embodiments as described, the cells lack expression of hTERT or telomerase.

In the embodiments herein, the cell population is a substantially homogeneous population of postpartum-derived cells. In a specific embodiment, the population is a homogeneous population of postpartum-derived cells. In embodiments, the postpartum-derived cells are derived from human umbilical cord tissue or placental tissue substantially free of blood. In embodiments herein, the cell population may be in a composition; in some embodiments, the composition may be a pharmaceutical composition comprising a pharmaceutically-acceptable carrier.

In certain embodiments, the population of postpartum-derived cells as described above is administered with at least one other cell type, such as an astrocyte, oligodendrocyte, neuron, neural progenitor, neural stem cell, retinal epithelial stem cell, corneal epithelial stem cell, or other multipotent or pluripotent stem cell. In these embodiments, the other cell type can be administered simultaneously with, before, or after, the cell population or the conditioned medium.

In these and other embodiments, the population of postpartum-derived cells as described above is administered with at least one other agent, such as a drug for ocular therapy, or another beneficial adjunctive agent such as an anti-inflammatory agent, anti-apoptotic agents, antioxidants or growth factors. In these embodiments, the other agent can be administered simultaneously with, before, or after, the cell population or the conditioned media.

In various embodiments, the population of postpartum-derived cells is administered to the surface of an eye, or is administered to the interior of an eye or to a location in proximity to the eye (e.g., behind the eye). The population of postpartum-derived cells can be administered by injection to the eye, such as subretinal injection, through a cannula or from a device implanted in the patient's body within or in proximity to the eye, or may be administered by implantation of a matrix or scaffold with the postpartum-derived cell population or conditioned media. In the embodiments herein, the population of postpartum-derived cells may be administered at various times, as a single point in time or at multiple points in time. In specific embodiments, the cells may be administered by injection as a single injection or more than one injection and at different points in time.

In certain embodiments, the composition or pharmaceutical composition is formulated for administration to the surface of an eye. Alternatively, they can be formulated for administration to the interior of an eye or in proximity to the eye (e.g., behind the eye). The compositions also can be formulated as a matrix or scaffold containing the postpartum-derived cells or conditioned media.

### BRIEF DESCRIPTION OF THE DRAWINGS

FIGS. 1A-1C hUTC inhibited choroidal neovascularization in vivo.

FIGS. 2A-2B hUTC reduced choroidal VEGF level in vivo.

FIGS. 3A-3D hUTC reduced VEGF in vitro. (FIG. 3A) ARPE19 cells were seeded at 60×10³ cells/well in 24-well plate and cultured with or without density-dependent hUTC for 48 hours followed by VEGF measurement by ELISA. (FIG. 3B) Conditioned media were collected from ARPE19 and hUTC cultured individually in 24-well plate, and then mixed at 1:1 ratio, and cultured for 5 minutes and 2 days, respectively at 37° C. followed by VEGF measurement. (FIG. 3C) Recombinant human VEGF (300 pg/mL) was added in hUTC CM and incubated for 5 minutes and 2 days, respectively at 37° C. followed by VEGF measurement. (FIG. 3D) Recombinant humanVEGF (300 pg/mL) was added in hUTC CM pre-incubated with a broad-spectrum protease inhibitor cocktail and cultured at 37° C. for 5 minutes. VEGF was then measured by ELISA. Data represent mean±SEM (n=3).

FIG. 4 Neutralizing antibody against sVEGFR1 recovers the VEGF level in hUTC conditioned medium. hUTC conditioned medium was pre-incubated with a neutralizing antibody (10 ug/mL) against sVEGFR1 for 1 hour at 37° C. followed by addition of rhVEGF at 1 ng/mL and incubated for another 30 minutes. VEGF level was measured by ELISA.

FIG. 5 Western blot analysis of sVEGFR1 in hUTC conditioned medium. The elutes of hUTC CM and control medium from VEGF pull-down assay were subject to SDS-PAGF. Briefly, 40 uL of tris-eluate solution mixed with sample buffer, and 25 ng of recombinant human soluble VEGFR1 were run on an SDS-PAGE gel and the gel resolved proteins were transferred onto a PVDF membrane. The membrane was blocked with 5% (W/V) BSA and subsequently probed with a biotinylated anti-VEGFR1 antibody targeting the extracellular region of VEGFR1. After washing, the membrane was incubated with streptavidin-HRP and immunoreactive bands were visualized with ECL. (CON, hUTC control medium sample; NEG: negative control (control medium); REC, recombinant human sVEGFR1.)

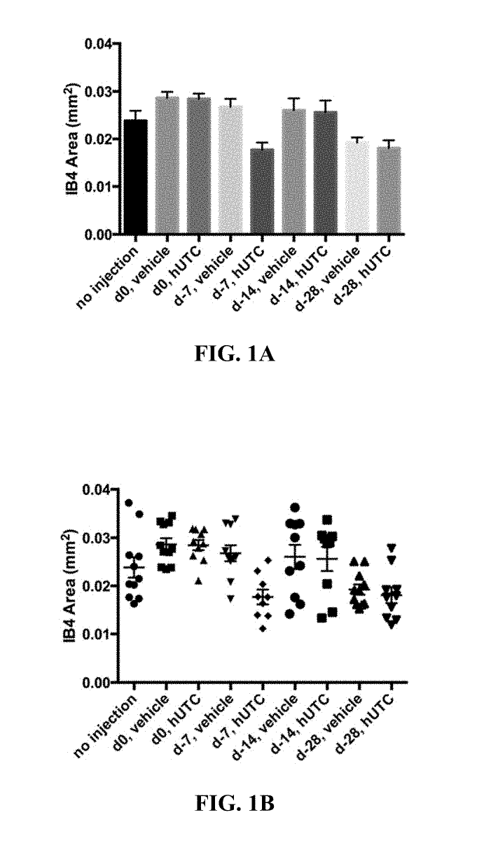

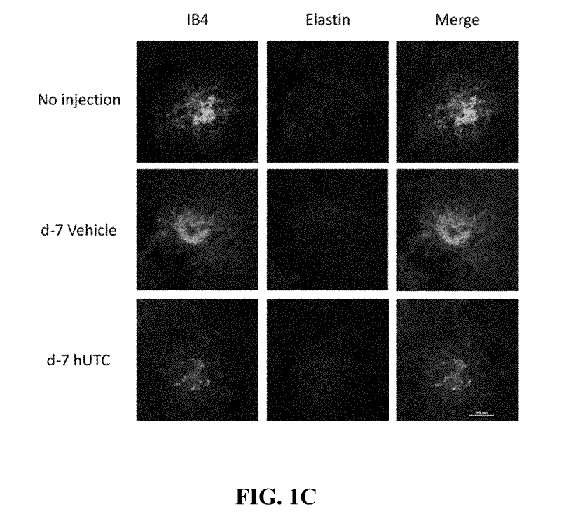

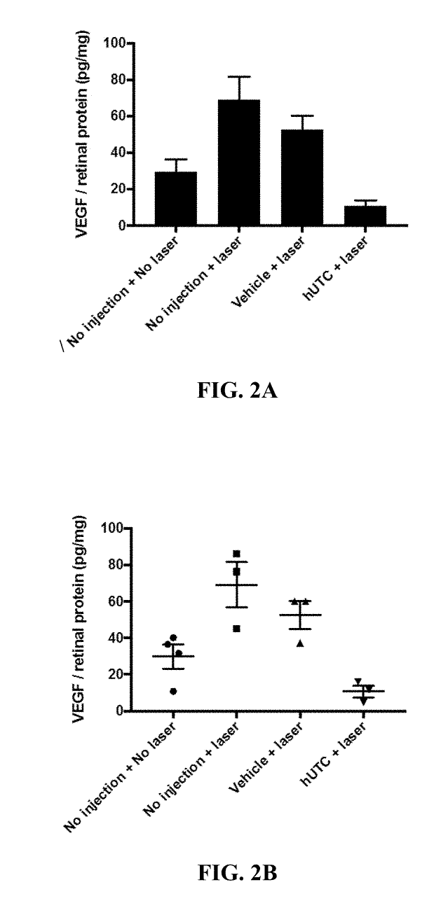

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical changes may be made without departing from the spirit or scope of the invention. To avoid detail not necessary to enable those skilled in the art to practice the embodiments described herein, the description may omit certain information known to those skilled in the art.

Progenitor cells isolated from postpartum tissue, and conditioned media derived from progenitor cells, such as cells isolated from postpartum umbilical cord or placenta, in accordance with methods known in the art provide a new source for treating ocular degenerative conditions. Accordingly, the various embodiments described herein feature methods and compositions for treating ophthalmic disease, including reducing retinal neovascularization using progenitor cells, such as postpartum-derived cells, and conditioned media produced from those cells.

### Definitions

Various terms used throughout the specification and claims are defined as set forth below and are intended to clarify the invention. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

Stem cells are undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts.

At the present time, stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. Induced pluripotent stem cells (iPS cells) are adult cells that are converted into pluripotent stem cells. (Takahashi et al., Cell, 2006; 126(4):663-676; Takahashi et al., Cell, 2007; 131:1-12). An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development). Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

The cells exemplified herein and preferred for use in the present invention are generally referred to as postpartum-derived cells (or PPDCs). They also may sometimes be referred to more specifically as umbilicus-derived cells (UDCs) or placenta-derived cells (PDCs). In addition, the cells may be described as being stem or progenitor cells, the latter term being used in the broad sense. The term derived is used to indicate that the cells have been obtained from their biological source and grown or otherwise manipulated in vitro (e.g., cultured in a Growth Medium to expand the population and/or to produce a cell line). The in vitro manipulations of umbilical stem cells and placental stem cells and unique features of the umbilicus-derived cells and placental-derived cells of the present invention are described in detail below. Cells isolated from postpartum placenta and umbilicus by other means are also considered suitable for use in the present invention. These other cells are referred to herein as postpartum cells (rather than postpartum-derived cells).

Various terms are used to describe cells in culture. Cell culture refers generally to cells taken from a living organism and grown under controlled conditions (“in culture” or “cultured”). A primary cell culture is a culture of cells, tissues, or organs taken directly from an organism(s) before the first subculture. Cells are expanded in culture when they are placed in a Growth Medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

The term growth medium generally refers to a medium sufficient for the culturing of PPDCs. In particular, one presently preferred medium for the culturing of the cells of an embodiment of the invention comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics ((preferably 50-100 Units/milliliter penicillin, 50-100 microgram/milliliter streptomycin, and 0-0.25 microgram/milliliter amphotericin B; Invitrogen, Carlsbad, Calif.)), and 0.001% (v/v) 2-mercaptoethanol (Sigma, St. Louis Mo.). As used in the Examples below, Growth Medium refers to DMEM-low glucose with 15% fetal bovine serum and antibiotics/antimycotics (when penicillin/streptomycin are included, it is preferably at 50 U/ml and 50 microgram/ml respectively; when penicillin/streptomycin/amphotericin are used, it is preferably at 100 U/ml, 100 microgram/ml and 0.25 microgram/ml, respectively). In some cases different growth media are used, or different supplementations are provided, and these are normally indicated in the text as supplementations to Growth Medium.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes survival, growth, differentiation, proliferation and/or maturation of a cell, or stimulates increased activity of a cell. The interaction between cells via trophic factors may occur between cells of different types. Cell interaction by way of trophic factors is found in essentially all cell types, and is a particularly significant means of communication among neural cell types. Trophic factors also can function in an autocrine fashion, i.e., a cell may produce trophic factors that affect its own survival, growth, differentiation, proliferation and/or maturation.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone.

The terms ocular, ophthalmic and optic are used interchangeably herein to define “of, or about, or related to the eye.” The term ocular degenerative condition (or disorder) is an inclusive term encompassing acute and chronic conditions, disorders or diseases of the eye, inclusive of the neural connection between the eye and the brain, involving cell damage, degeneration or loss. An ocular degenerative condition may be age-related, or it may result from injury or trauma, or it may be related to a specific disease or disorder. Acute ocular degenerative conditions include, but are not limited to, conditions associated with cell death or compromise affecting the eye including conditions arising from cerebrovascular insufficiency, focal or diffuse brain trauma, diffuse brain damage, infection or inflammatory conditions of the eye, retinal tearing or detachment, intra-ocular lesions (contusion penetration, compression, laceration) or other physical injury (e.g., physical or chemical burns). Chronic ocular degenerative conditions (including progressive conditions) include, but are not limited to, retinopathies and other retinal/macular disorders such as retinitis pigmentosa (RP), age-related macular degeneration (AMD), choroidal neovascular membrane (CNVM); retinopathies such as diabetic retinopathy, occlusive retinopathy, sickle cell retinopathy and hypertensive retinopathy, central retinal vein occlusion, stenosis of the carotid artery, optic neuropathies such as glaucoma and related syndromes; disorders of the lens and outer eye, e.g., limbal stem cell deficiency (LSCD), also referred to as limbal epithelial cell deficiency (LECD), such as occurs in chemical or thermal injury, Steven-Johnson syndrome, contact lens-induced keratopathy, ocular cicatricial pemphigoid, congenital diseases of aniridia or ectodermal dysplasia, and multiple endocrine deficiency-associated keratitis.

The term treating (or treatment of) an ocular degenerative condition refers to ameliorating the effects of, or delaying, halting or reversing the progress of, or delaying or preventing the onset of, an ocular degenerative condition as defined herein.

The term effective amount refers to a concentration or amount of a reagent or pharmaceutical composition, such as a growth factor, differentiation agent, trophic factor, cell population or other agent, that is effective for producing an intended result, including cell growth and/or differentiation in vitro or in vivo, or treatment of ocular degenerative conditions, as described herein. With respect to growth factors, an effective amount may range from about 1 nanogram/milliliter to about 1 microgram/milliliter. With respect to PPDCs as administered to a patient in vivo, an effective amount may range from as few as several hundred or fewer, to as many as several million or more. In specific embodiments, an effective amount may range from 10³ to 11¹¹, more specifically at least about 10⁴ cells. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term patient or subject refers to animals, including mammals, preferably humans, who are treated with the cells or pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

Several terms are used herein with respect to cell replacement therapy. The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy. Transplantation as used herein refers to the introduction of autologous, or allogeneic donor cell replacement therapy into a recipient.

Description

Ocular degenerative conditions, which encompass acute, chronic and progressive disorders and diseases having divergent causes, have as a common feature the dysfunction or loss of a specific or vulnerable group of ocular cells. This commonality enables development of similar therapeutic approaches for the repair or regeneration of vulnerable, damaged or lost ocular tissue or cells, one of which is cell-based therapy. Development of cell therapy for ocular degenerative conditions has been limited to a comparatively few types of stem or progenitor cells, including ocular-derived stem cells themselves (e.g., retinal and corneal stem cells), embryonic stem cells and a few types of adult stem or progenitor cells (e.g., neural, mucosal epithelial and bone marrow stem cells). Cells isolated from the postpartum umbilical cord and placenta have been identified as a significant new source of progenitor cells for this purpose. (U.S. 2005/0037491 and U.S. 2010/0272803). Moreover, conditioned media generated from cells isolated from the postpartum placenta and umbilical cord tissue provides another new source for treating ocular degenerative conditions. Accordingly, in its various embodiments described herein, the present invention features methods and pharmaceutical compositions for (repair and regeneration of ocular tissues), which use conditioned media from progenitor cells, such as cells isolated from postpartum umbilical cord or placenta. The invention is applicable to ocular degenerative conditions, but is expected to be particularly suitable for a number of ocular disorders for which treatment or cure has been difficult or unavailable. These include, without limitation, retinopathies, such as age-related macular degeneration, retinitis pigmentosa, diabetic retinopathy, neovascularization, choroidal neovascularization, and angiogenesis.

Conditioned media derived from progenitor cells, such as cells isolated from postpartum umbilical cord or placenta, in accordance with any method known in the art is expected to be suitable for use in the present invention. In one embodiment, however, the invention uses conditioned media derived from umbilical cord tissue-derived cells (hUTCs) or placental-tissue derived cells (PDCs) as defined above, which are derived from umbilical cord tissue or placenta that has been rendered substantially free of blood, preferably in accordance with the method set forth below. The hUTCs or PDCs are capable of expansion in culture and have the potential to differentiate into cells of other phenotypes. Certain embodiments feature conditioned media prepared from such progenitor cells, pharmaceutical compositions comprising the conditioned media, and methods of using the pharmaceutical compositions for treatment of patients with acute or chronic ocular degenerative conditions. The postpartum-derived cells of the present invention have been characterized by their growth properties in culture, by their cell surface markers, by their gene expression, by their ability to produce certain biochemical trophic factors, and by their immunological properties. The conditioned media derived from the postpartum-derived cells have been characterized by the trophic factors secreted by the cells.

The cells, cell populations and preparations comprising cell lysates, conditioned media and the like, used in the compositions and methods of the present invention are described herein, and in detail in U.S. Pat. Nos. 7,524,489, and 7,510,873, and U.S. Pub. App. No. 2005/0058631, each incorporated by reference herein.

Characteristics of Cells

The progenitor cells of the invention, such as PPDCs, may be characterized, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

Examples of PPDCs derived from umbilicus tissue were deposited with the American Type Culture Collection on (ATCC, 10801 University Boulevard, Manassas, Va., 20110) Jun. 10, 2004, and assigned ATCC Accession Numbers as follows: (1) strain designation UMB 022803 (P7) was assigned Accession No. PTA-6067; and (2) strain designation UMB 022803 (P17) was assigned Accession No. PTA-6068. Examples of PPDCs derived from placental tissue were deposited with the ATCC (Manassas, Va.) and assigned ATCC Accession Numbers as follows: (1) strain designation PLA 071003 (P8) was deposited Jun. 15, 2004 and assigned Accession No. PTA-6074; (2) strain designation PLA 071003 (P11) was deposited Jun. 15, 2004 and assigned Accession No. PTA-6075; and (3) strain designation PLA 071003 (P16) was deposited Jun. 16, 2004 and assigned Accession No. PTA-6079.

In various embodiments, the PPDCs possess one or more of the following growth features: (1) they require L-valine for growth in culture; (2) they are capable of growth in atmospheres containing oxygen from about 5% to at least about 20%; (3) they have the potential for at least about 40 doublings in culture before reaching senescence; and (4) they attach and expand on a coated or uncoated tissue culture vessel, wherein the coated tissue culture vessel comprises a coating of gelatin, laminin, collagen, polyomithine, vitronectin or fibronectin.

In certain embodiments the PPDCs possess a normal karyotype, which is maintained as the cells are passaged. Karyotyping is particularly useful for identifying and distinguishing neonatal from maternal cells derived from placenta. Methods for karyotyping are available and known to those of skill in the art.

In other embodiments, the PPDCs may be characterized by production of certain proteins, including: (1) production of at least one of vimentin and alpha-smooth muscle actin; and (2) production of at least one of CD10, CD13, CD44, CD73, CD90, PDGFr-alpha, PD-L2 and HLA-A,B,C cell surface markers, as detected by flow cytometry. In other embodiments, the PPDCs may be characterized by lack of production of at least one of CD31, CD34, CD45, CD80, CD86, CD117, CD141, CD178, B7-H2, HLA-G, and HLA-DR,DP,DQ cell surface markers, as detected by flow cytometry. Particularly preferred are cells that produce vimentin and alpha-smooth muscle actin.

In other embodiments, the PPDCs may be characterized by gene expression, which relative to a human cell that is a fibroblast, a mesenchymal stem cell, or an iliac crest bone marrow cell, is increased for a gene encoding at least one of interleukin 8; reticulon 1; chemokine (C--X--C motif) ligand 1 (melonoma growth stimulating activity, alpha); chemokine (C--X--C motif) ligand 6 (granulocyte chemotactic protein 2); chemokine (C--X--C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113. In an embodiment, the PPDCs derived from umbilical cord tissue may be characterized by gene expression, which relative to a human cell that is a fibroblast, a mesenchymal stem cell, or an iliac crest bone marrow cell, is increased for a gene encoding at least one of interleukin 8; reticulon 1; or chemokine (C--X--C motif) ligand 3. In another embodiment, the PPDCs derived from placental tissue may be characterized by gene expression, which relative to a human cell that is a fibroblast, a mesenchymal stem cell, or an iliac crest bone marrow cell, is increased for a gene encoding at least one of renin or oxidized low density lipoprotein receptor 1.

In yet other embodiments, the PPDCs may be characterized by gene expression, which relative to a human cell that is a fibroblast, a mesenchymal stem cell, or an iliac crest bone marrow cell, is reduced for a gene encoding at least one of: short stature homeobox 2; heat shock 27 kDa protein 2; chemokine (C--X--C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAAI034 protein; vesicle-associated membrane protein 5 (myobrevin); EGF-containing fibulin-like extracellular matrix protein 1; early growth response 3; distal-less homeo box 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle).

In other embodiments, the PPDCs derived from umbilical cord tissue may be characterized by secretion of trophic factors selected from thrombospondin-1, thrombospondin-2, and thrombospondin-4. In embodiments, the PPDCs may be characterized by secretion of at least one of MCP-1, IL-6, IL-8, GCP-2, HGF, KGF, FGF, HB-EGF, BDNF, TPO, MIP1b, 1309, RANTES, MDC, and TIMP1. In some embodiments, the PPDCs derived from umbilical cord tissue may be characterized by lack of secretion of at least one of TGF-beta2, ANG2, PDGFbb, MIP1a and VEGF, as detected by ELISA. In alternative embodiments, PPDCs derived from placenta tissue may be characteristics by secretion of at least one of MCP-1, IL-6, IL-8, GCP-2, HGF, KGF, HB-EGF, BDNF, TPO, MIP1a, RANTES, and TIMP1, and lack of secretion of at least one of TGF-beta2, ANG2, PDGFbb, FGF, and VEGF, as detected by ELISA. In further embodiments, the PPDCs lack expression of hTERT or telomerase.

In preferred embodiments, the cell comprises two or more of the above-listed growth, protein/surface marker production, gene expression or substance-secretion characteristics. More preferred are those cells comprising, three, four, or five or more of the characteristics. Still more preferred are PPDCs comprising six, seven, or eight or more of the characteristics. Still more preferred presently are those cells comprising all of above characteristics.

Conditioned Medium

In one aspect, the invention provides conditioned medium from cultured progenitor cells, such as postpartum-derived cells, or other progenitor cells, for use in vitro and in vivo as described below. Use of such conditioned medium allows the beneficial trophic factors secreted by the cells to be used allogeneically in a patient without introducing intact cells that could trigger rejection, or other adverse immunological responses. Conditioned medium is prepared by culturing cells (such as a population of cells) in a culture medium, then removing the cells from the medium. In certain embodiments, the postpartum cells are UTCs or PDCs, more preferably hUTCs.

Conditioned medium prepared from populations of cells as described above may be used as is, further concentrated, by for example, ultrafiltration or lyophilization, or even dried, partially purified, combined with pharmaceutically-acceptable carriers or diluents as are known in the art, or combined with other compounds such as biologicals, for example pharmaceutically useful protein compositions. Conditioned medium may be used in vitro or in vivo, alone or for example, with autologous or syngeneic live cells. The conditioned medium, if introduced in vivo, may be introduced locally at a site of treatment, or remotely to provide, for example needed cellular growth or trophic factors to a patient.

Previously, it has been demonstrated that human umbilical cord tissue-derived cells improved visual function and ameliorated retinal degeneration (US 2010/0272803). It also has been demonstrated that postpartum-derived cells can be used to promote photoreceptor rescue and thus preserve photoreceptors in a RCS model. (US 2010/0272803). Injection of hUTC subretinally into RCS rat eye improved visual acuity and ameliorated retinal degeneration. Moreover, treatment with conditioned medium (CM) derived from hUTC restored phagocytosis of ROS in dystrophic RPE cells in vitro. (US 2010/0272803). Here, embodiments of the invention disclose the positive effect of hUTCs to inhibit or reduce neovascularization.

Pharmaceutical Compositions

In another aspect, the invention provides pharmaceutical compositions that use non-embryronic stem cells such as postpartum cells (preferably PPDCs), cell populations thereof, conditioned media produced by such cells, and cell components and products produced by such cells in various methods for treatment of ocular degenerative conditions. Certain embodiments encompass pharmaceutical compositions comprising live cells (e.g., PPDCs alone or admixed with other cell types). Other embodiments encompass pharmaceutical compositions comprising PPDC conditioned medium. Additional embodiments may use cellular components of PPDC (e.g., cell lysates, soluble cell fractions, ECM, or components of any of the foregoing) or products (e.g., trophic and other biological factors produced naturally by the cells or through genetic modification, conditioned medium from culturing the cells). In either case, the pharmaceutical composition may further comprise other active agents, such as anti-inflammatory agents, anti-apoptotic agents, antioxidants, growth factors, neurotrophic factors or neuroregenerative, neuroprotective or ophthalmic drugs as known in the art.

Pharmaceutical compositions of the invention comprise progenitor cells, such as postpartum cells (preferably PPDCs), conditioned media generated from those cells, or components or products thereof, formulated with a pharmaceutically acceptable carrier or medium. Suitable pharmaceutically acceptable carriers include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Typically, but not exclusively, pharmaceutical compositions comprising cellular components or products, but not live cells, are formulated as liquids. Pharmaceutical compositions comprising PPDC live cells are typically formulated as liquids, semisolids (e.g., gels) or solids (e.g., matrices, scaffolds and the like, as appropriate for ophthalmic tissue engineering).

Formulations for injection are preferably designed for single-use administration and do not contain preservatives. Injectable solutions should have isotonicity equivalent to 0.9% sodium chloride solution (osmolality of 290-300 milliosmoles). This may be attained by addition of sodium chloride or other co-solvents as listed above, or excipients such as buffering agents and antioxidants, as listed above.

The tissues of the anterior chamber of the eye are bathed by the aqueous humor, while the retina is under continuous exposure to the vitreous. These fluids/gels exist in a highly reducing redox state because they contain antioxidant compounds and enzymes. Therefore, it may be advantageous to include a reducing agent in the ophthalmologic compositions. Suitable reducing agents include N-acetylcysteine, ascorbic acid or a salt form, and sodium sulfite or metabisulfite, with ascorbic acid and/or N-acetylcysteine or glutathione being particularly suitable for injectable solutions.

Pharmaceutical compositions comprising cells or conditioned medium, or cell components or cell products may be delivered to the eye of a patient in one or more of several delivery modes known in the art. In one embodiment that may be suitable for use in some instances, the compositions are topically delivered to the eye in eye drops or washes. In another embodiment, the compositions may be delivered to various locations within the eye via periodic intraocular injection or by infusion in an irrigating solution such as BSS or BSS PLUS (Alcon USA, Fort Worth, Tex.). Alternatively, the compositions may be applied in other ophthalmologic dosage forms known to those skilled in the art, such as pre-formed or in situ-formed gels or liposomes, for example as disclosed in U.S. Pat. No. 5,718,922 to Herrero-Vanrell. In another embodiment, the composition may be delivered to or through the lens of an eye in need of treatment via a contact lens (e.g. Lidofilcon B, Bausch & Lomb CW79 or DELTACON (Deltafilcon A) or other object temporarily resident upon the surface of the eye. In other embodiments, supports such as a collagen corneal shield (e.g. BIO-COR dissolvable corneal shields, Summit Technology, Watertown, Mass.) can be employed. The compositions can also be administered by infusion into the eyeball, either through a cannula from an osmotic pump (ALZET, Alza Corp., Palo Alto, Calif.) or by implantation of timed-release capsules (OCCUSENT) or biodegradable disks (OCULEX, OCUSERT). These routes of administration have the advantage of providing a continuous supply of the pharmaceutical composition to the eye. This may be an advantage for local delivery to the cornea.

Abbreviations

The following abbreviations may appear in the examples and elsewhere in the specification and claims: ANG2 (or Ang2) for angiopoietin 2; APC for antigen-presenting cells; BDNF for brain-derived neurotrophic factor; bFGF for basic fibroblast growth factor; CK18 for cytokeratin 18; CNS for central nervous system; CNTF for ciliary neurotrophic factor; CXC ligand 3 for chemokine receptor ligand 3; DMEM for Dulbecco's Minimal Essential Medium; DMEM:lg (or DMEM:Lg, DMEM:LG) for DMEM with low glucose; EDTA for ethylene diamine tetraacetic acid; EGF (or E) for epidermal growth factor; FACS for fluorescent activated cell sorting; FBS for fetal bovine serum; FGF (or F) for fibroblast growth factor; GBP for gabapentin; GCP-2 for granulocyte chemotactic protein-2; GDNF for glial cell-derived neurotrophic factor; GFAP for glial fibrillary acidic protein; HB-EGF for heparin-binding epidermal growth factor; HCAEC for Human coronary artery endothelial cells; HGF for hepatocyte growth factor; hMSC for Human mesenchymal stem cells; HNF-1alpha for hepatocyte-specific transcription factor; HVVEC for Human umbilical vein endothelial cells; 1309 for a chemokine and the ligand for the CCR8 receptor; IGF-1 for insulin-like growth factor 1; IL-6 for interleukin-6; IL-8 for interleukin 8; K19 for keratin 19; K8 for keratin 8; KGF for keratinocyte growth factor; LIF for leukemia inhibitory factor; MBP for myelin basic protein; MCP-1 for monocyte chemotactic protein 1; MDC for macrophage-derived chemokine; MIPlalpha for macrophage inflammatory protein 1 alpha; MIP1beta for macrophage inflammatory protein 1 beta; MMP for matrix metalloprotease (MMP); MSC for mesenchymal stem cells; NHDF for Normal Human Dermal Fibroblasts; NPE for Neural Progenitor Expansion media; NT3 for neurotrophin 3; 04 for oligodendrocyte or glial differentiation marker 04; PBMC for Peripheral blood mononuclear cell; PBS for phosphate buffered saline; PDGF-CC for platelet derived growth factor C; PDGF-DD for platelet derived growth factor D; PDGFbb for platelet derived growth factor bb; PO for “per os” (by mouth); PNS for peripheral nervous system; Rantes (or RANTES) for regulated on activation, normal T cell expressed and secreted; rhGDF-5 for recombinant human growth and differentiation factor 5; SC for subcutaneously; SDF-1alpha for stromal-derived factor 1 alpha; SHH for sonic hedgehog; SOP for standard operating procedure; TARC for thymus and activation-regulated chemokine; TCP for Tissue culture plastic; TCPS for tissue culture polystyrene; TGFbeta1 for transforming growth factor beta1; TGFbeta2 for transforming growth factor beta2; TGF beta-3 for transforming growth factor beta-3; TIMP1 for tissue inhibitor of matrix metalloproteinase 1; TPO for thrombopoietin; TSP for thrombospondin; TUJ1 for BIII Tubulin; VEGF for vascular endothelial growth factor; vWF for von Willebrand factor; and alphaFP for alpha-fetoprotein.

### EXAMPLES

The following examples are provided to describe the invention in greater detail. They are intended to illustrate, not to limit, the invention.

### Example 1

### Effect of Human Umbilical Tissue-Derived Cells on Neovascularization

The effect of human umbilical tissue-derived cells on neovascularization and VEGF was examined.

Materials

VEGF ELISA kit was from Thermo Scientific (Pittsburgh, Pa.). sVEGFR1 and rat VEGF ELISA kits were from R&D Systems, Inc. (Minneapolis, Minn.). Recombinant human VEGF165 (a 165 amino acid splice variant of VEGF) was from EMD Chemicals (Gibbstown, N.J.). Halt Protease inhibitor Single-Use Cocktail was from Thermo Scientific (Pittsburgh, Pa.), and used at 1×, or 3× of the concentrations as instructed by the vendor. Anti-human VEGFR1 antibodies (AF321, BAF321) and normal goat IgG isotype control antibody were from R&D Systems (Minneapolis, Minn.). Recombinant human sVEGFR1 was from Cell Science (Canton, Mass.).

Methods

Animals and Treatments:

All procedures were performed with strict adherence to guidelines for animal use and experimentation.

Sub-Retinal Injections:

Six-week old male Brown Norway rats (Charles River; Wilmington, Mass.) were anesthetized with an intraperitoneal injection (IP) of ketamine (80 mg/kg)/xylazine (8 mg/kg) (Henry Schein; Melville, N.Y.). Their pupils were dilated with 1% tropicamide (Alcon Pharmaceuticals; Ft. Worth, Tex.); they were placed in lateral recumbency under a Zeiss Operating Microscope (Zeiss; Peabody, Mass.) and the head was immobilized by holding it with one hand. GenTeal® lubricant eye gel (Alcon Pharmaceuticals) was applied to the corneal surface and the fundus was visualized. A guide hole for the delivery of hUTC was prepared as follows: while proptosing the eye, the globe was punctured with a 19°-beveled 30 G needle (Hamilton®; Reno, Nev.) immediately posterior to the corneal limbus at a steep angle to avoid touching the lens. The needle was retracted while keeping the head immobilized. A Hamilton syringe fitted with a 33 G blunt-ended needle (Hamilton®) was pre-loaded with 2 μl of an hUTC suspension. This needle was inserted into the guide hole at a steep angle to avoid contact with the lens and advanced through the eye until penetration of the retina and entrance into the subretinal space. The syringe was held in place by one operator while another slowly delivered its contents into the subretinal space, creating a visible retinal detachment. Following subretinal delivery, the needle was gently withdrawn. Immediately following the injection procedure, 2-3 drops of a 0.3% Tobramycin Ophthalmic Solution USP (Allivet; Miami, Fla.) was applied to the anterior surface of eye to prevent infection.

Laser-Induced Choroidal Neovascularization:

Six-week old Adult Brown-Norway rats (Charles River) were anesthetized with an IP injection of ketamine (80 mg/Kg)/(8 mg/kg) xylazine (Henry Schein). The pupils were dilated with 1% tropicamide (Alcon). Using a hand-held cover slip as a contact lens, and GenTeal® lubricating eye gel (Alcon) as a medium contacting the cover slip and the service of the cornea, an AC-2000 argon laser photocoagulator operating at 488/514 nm (NIDEK INC., Fremont Calif.) coupled to a SL-1600 slit-lamp (NIDEK INC.), was used to create four burns equidistant from the optic nerve head in the retinal mid-pheriphery. Lesions were created with laser parameters that included 100 μm spot size, 0.1 sec duration, and 120 mW.

Choroidal Mounts and Tissue Staining:

Choroidal mounts and tissue staining were performed in a manner similar to that described by Bora et al. (2005). Animals were deeply anesthetized and euthanized by cervical dislocation. The eyes were then enucleated and placed in 10% formalin (Sigma) for 2 hr. The retinal pigment epithelium (RPE)-choroid-sclera complexes were obtained by hemisecting the eyes, removing the lens, and peeling the neural retina away from the underlying RPE. At least four radial cuts were made to allow this tissue to be flattened. Constituent endothelial cells of choroidal neovascular lesions were identified with FITC-conjugated Griffonia simplicifolia isolectin B4 (Sigma) while the elastin of the extracellular matrix was identified using goat anti-elastin antibody conjugated to Cy3 (Santa Cruz Biotech., Inc; Santa Cruz, Calif.). The flat-mount was then placed onto a microscope slide with the RPE-side facing up. Gel Mount media (Biomedia; Victoria, Australia) was applied to the tissue before covering the slide with a cover slip. Choroidal mounts were visualized using the 10× objective of an epifluorescent compound microscope fitted with the appropriate excitation and emission filters (Provis AX-70, Olympus; Japan). Images of the neovascular lesions were captured using a digital camera attached to the Provis system (DP71, Olympus; Japan) using image capture software (DP Controller, Olympus; Japan). Image sizes were calibrated using the DP Controller's automated scaling feature.

Choroidal VEGF Level Examination:

Laser-induced rupture of Bruch's membrane was used to generate choroidal neovascularization in 6-week old, male Brown Norway rats. Rats were divided into treatment arms that included non-laser control, or laser plus no injection, vehicle injection, and hUTC injection administered 7 days pre-laser. Choroidal tissues were dissected at 3 days post-laser, and VEGF protein was measured using a rat VEGF ELISA (R&D Systems; RRV00). VEGF protein levels were normalized to total protein in the choroidal tissues. Collected tissues included choroid, Bruch's membrane, and RPE.

hUTC Culture:

hUTC were isolated, cultured and cryopreserved as described below in Examples 2-8, and as previously described in U.S. Pat. Nos. 7,524,489, and 7,510,873, U.S. Pub. App. No. 2005/0058631, U.S. Patent Application No. 62/358,389, filed Jul. 5, 2016, and U.S. Patent Application No. 62/514,317, filed Jun. 2, 2017, all of which are incorporated by reference in their entirety. Briefly, human umbilical cords were obtained with donor consent following live births from the National Disease Research Interchange (Philadelphia, Pa.). Tissues were minced and enzymatically digested. After almost complete digestion with a Dulbecco's modified Eagle's medium (DMEM)-low glucose (Lg) (Invitrogen, Carlsbad, Calif.) medium containing a mixture of 50 U/mL collagenase (Sigma, St. Louis), hyaluronidase and neutral protease, the cell suspension was filtered through a 70 μm filter, and the supernatant was centrifuged at 350 g. Isolated cells were washed in DMEM-Lg a few times and seeded at a density of 5,000 cells/cm2 in DMEM-Lg medium containing 15% (v/v) FBS (Hyclone, Logan, Utah) and 4 mM L-glutamine (Gibco, Grand Island, N.Y.). When cells reached approximately 70% confluence, they were passaged using TrypLE (Gibco, Grand Island, N.Y.). Cells were harvested after several passages and banked.

Preparation of hUTC and ARPE19 Conditioned Medium:

On day 1, hUTC or ARPE19 were thawed and plated in 6-well plate at 0.288×106 cells/2.4 ml/well in DMEM-Lg medium containing 15% (v/v) FBS (Hyclone, Logan, Utah) and 4 mM L-glutamine (Gibco, Grand Island, N.Y.). On day 2, medium was aspirated and replenished with DMEM/F12 medium (ATCC) containing 10% v/v FBS and 50 U/ml penicillin (Invitrogen), 50 μg/ml Streptomycin (Invitrogen). Cells continued to culture for 48 hours. On day 4, CM from hUTC or ARPE19 was collected to be used for experiments directly, or frozen at −70° C. for future use.

Immunoprecipitation:

10 μg recombinant human VEGF165 (Peprotech, Princeton, N.J.) per mg Dynabeads (ThermoFisher Scientific, Rochester, N.Y.) was conjugated per manufacturer's directions for a final concentration of 10 mg beads/ml (100 μg VEGF/ml). 15 mL hUTC conditioned medium or control medium was mixed with 0.3 mg of VEGF-conjugated beads solution. Samples were incubated by rolling for 30 minutes at 4° C. and washed with a buffer (100 mM Tris, pH 7.4; 100 mM NaCl, 100 mM KCl, 1 mM MgCl2, 1 mM CaCl2, 1 mM EDTA buffer, 1% protease inhibitors). Samples were then eluted in 50 μl of 50 mM HCL for 5 minutes, and the eluates were finally mixed with 5 μl of 1.0 M Tris pH 8.5. For western blot analysis, 40 uL of Tris-eluate solution mixed with NuPAGE® LDS Sample Buffer (4×) (Life Technologies, Carlsbad, Calif.) and 25 ng of recombinant human soluble VEGFR1 were run on an SDS-PAGE gel and the gel resolved proteins were transferred onto a polyvinylidene difluoride (PVDF) membrane. The membrane was blocked with 5% (W/V) BSA and subsequently probed with a biotinylated anti-VEGFR1 antibody (R&D Systems, Minneapolis, Minn.) targeting the extracellular region of VEGFR1. After washing, the membrane was incubated with streptavidin-HRP (Jackson Immunoresearch, West Grove, Pa.) and immunoreactive bands were visualized with enhance chemiluminescence western blotting substrate. The remaining eluate was used for mass spectrometry analysis to identify VEGF-binding factors in hUTC conditioned medium.

Mass Spectrometry:

Two immunoprecipitation pull-down samples, prepared from hUTC conditioned medium and control medium (medium alone), were processed for mass spectrometry (MS) analysis. The samples were concentrated, reduced, alkylated, and digested. MS requisition was then done using peptide digestions of binding fractions followed by peptide mapping and data analysis.

R&a Peptide Digestion:

The samples (˜150 μL each) were concentrated to approximately 15 μL in a vacuum centrifuge. They were reduced (R) by mixing with 2 μL of 100 mM DDT; after which the solution was incubated at 60° C. for 30 min. After reduction, the samples were alkylated (A) by adding 2 μL of 100 mM IAA and incubating at RT in the dark for 20 min. Finally, a 1.0-4, aliquot of trypsin ([C]=0.5 μg/μL) was added and the solutions were digested overnight (˜16 h) at 37° C.

HPLC-MS Data Generation:

The peptide digests were analyzed using a Dionex U3000 HPLC coupled with a Bruker Daltonics micrOTOF-Q II mass spectrometer. An 18-4, aliquot of sample was injected for each analysis. The digests were separated using an LC Packings Acclaim PepMap C18, 180 μm×150 mm, 3 μm dₚ column; solvent A was 0.1% formic acid (FA) in water and solvent B was acetonitrile. The column was maintained at a temperature of 65° C. The flow rate was 6.0 μL/min, and the flow passed directly into the micrOTOF mass spectrometer. The electrospray emitter was held at ground, the transfer capillary voltage was −4.5 kV, and the end plate offset voltage was set at −500 V. One analysis was performed per sample: top-5 data directed analysis (DDA) with precursors selected from the mass range m/z 400-1700.

Data Analysis:

The results were searched against the NCBI non-redundant database (02° C.T11 release) with human taxonomy. Identified proteins listed in Table 1 were made from peptide MS/MS data with a significance of p<0.05 and the expectation threshold set at <0.05.

Statistical Analysis:

Statistical significance was assessed by unpaired two-tailed Student's t-test. A P value<0.05 was considered statistically significant. All statements of variability are for Standard Error of the Mean (SEM) unless noted otherwise.

Results

hUTC Reduced VEGF Release from ARPE-19 Cells In Vitro:

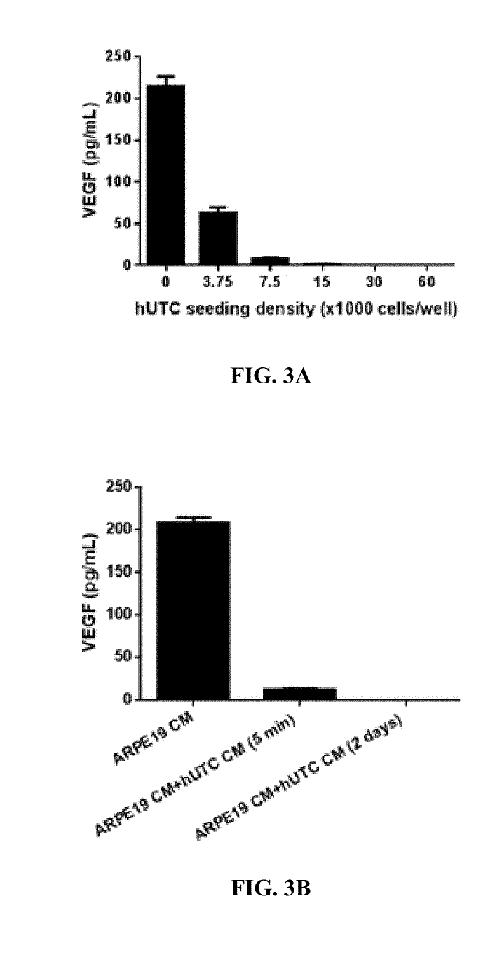

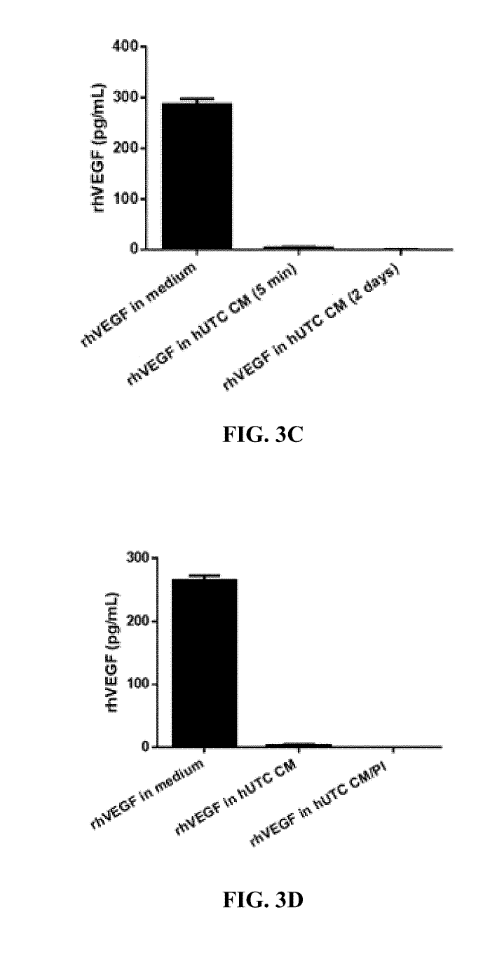

ARPE-19 cells were cultured with or without dose-dependently seeded hUTC to examine the effect of hUTC on RPE VEGF production in vitro. VEGF was secreted at around 220 pg/ml from ARPE-19 cultured alone 2-day post seeding, decreased dramatically from ARPE19 co-cultured with hUTC, and became completely undetectable wherein high density (30×10³ or 60×10³ cells/well) of hUTC were seeded (FIG. 3A). Moreover, when ARPE19 CM was incubated with hUTC CM, the VEGF in ARPE19 CM was reduced markedly and rapidly in 5 minutes, and became completely undetectable in 2 days (FIG. 3B). The level of VEGF in hUTC CM is under the detection limit (data not shown). Similarly, recombinant human VEGF (rhVEGF), when added in hUTC CM at 300 pg/mL, became negligible after 5 min incubation and undetectable in 2 days (FIG. 3C).

To examine the possibility of proteolytic degradation of VEGF, hUTC CM was pretreated with a broad spectrum of protease inhibitor cocktail for 30 minutes before rhVEGF (300 pg/ml) was added and incubated for 5 minutes. However, the pre-incubation of hUTC CM with protease inhibitor cocktail had no effect on the decrease of rhVEGF (FIG. 3D). Similarly, rhVEGF was under detectable level in hUTC CM with or without protease inhibitor cocktail after further incubation for 15, 30, 60 or 120 minutes (data not shown).

Identification of VEGF-Binding Factor(s) in hUTC Conditioned Medium

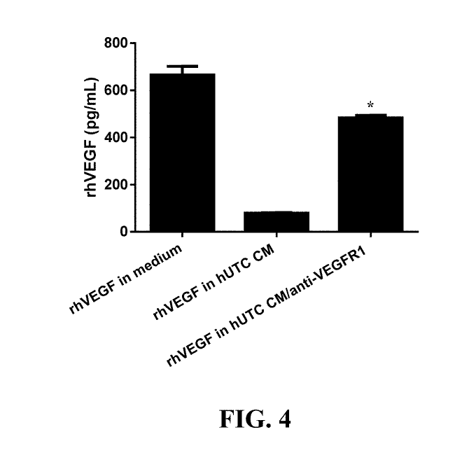

Many factors have been reported to bind to VEGF, such as neuropilin-1 and -2, thrombospondin, connective tissue growth factor, platelet factor-4, alpha 2-macroglobulin, collagen, and heparin sulphage proteoglycan. Immunoprecipitation fractions were prepared from hUTC CM and control medium using VEGF “pull-down” assay. Proteins in hUTC CM that bound to VEGF were pulled down by the rhVEGF conjugated-beads. The protein-beads complex was then eluted in buffers. Part of the eluate was used for western blot analysis, and the remaining eluate was further processed for mass spectrometry (MS) analysis. MS requisition was done using peptide digestions of binding fractions followed by peptide mapping and data analysis. The identified proteins in hUTC CM and control medium are summarized in Table 1. The nine peptides identified demonstrate the presence of the VEGFR1 protein in the hUTC conditioned media, but not in the control medium. No other proteins were identified with high confidence. We further confirmed this by the detection of high level of sVEGFR1 (1739.0±48.505 pg/mL) in hUTC CM using ELISA. The level of rhVEGF in human CM was significantly recovered when the CM was pre-incubated with a neutralizing antibody against sVEGFR1 (FIG. 4).

TABLE 1
Proteins identified in the submitted samples.

| | | | | Number |
|---|---|---|---|---|
| | MS | Accession | Identified | of |
| Sample | Data File | Number | Proteins | Peptides^{a} |
| Fresh | SCP_U03Q04_ | gi\|999627 | Trypsin, | 3 |
| Media | 07OCT11_04 | | porcine | |
| Conditioned | SCP_U03Q04_ | gi\|229892300 | Vascular | |
| Media | 07OCT11_05 | | endothelial | 9 |
| | | | growth | |
| | | | factor receptor 1 | |
| | | | [Homo sapiens] | |
| | | gi\|999627 | Trypsin, porcine | 3 |

^{a}Two or more peptides are required for high confidence protein identification.

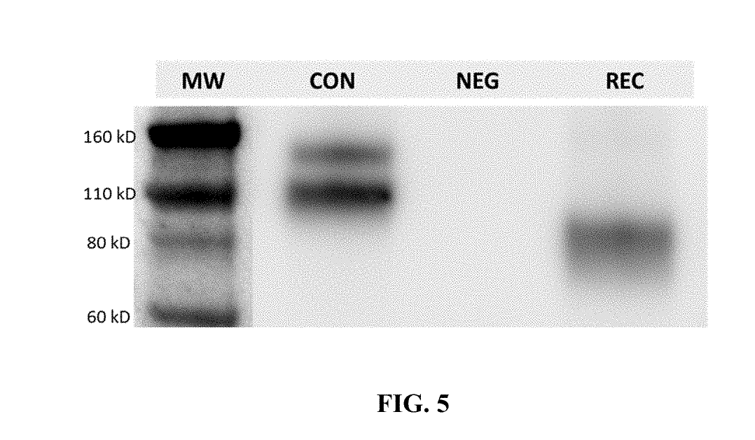

In addition, the hUTC CM elute from VEGF pull-down immunoprecipitation was subject to western blot analysis, and the molecular weight of sVEGFR1 in hUTC CM was evaluated. Recombinant human sVEGFR1, with a mass of approximately 96 kDa, was used as a positive control. The antibody to sVEGFR1 yielded a predominant band of ˜110 kDa and a minor band of ˜150 kDa in hUTC CM sample. A single band of ˜96 kDa was detected for recombinant human sVEGFR1 (Figure. 5). The molecular weight of sVEGFR1 has been reported to be both glycosylation- and species-dependent. The size of sVEGFR1 has been documented as 60 kDa in mice, 110 kDa when expressed by human umbilical vein endothelial cells and primary human dermal microvascular endothelial cells, 120-130 kDa when produced by melanoma cells, and 116 kDa in human placental tissue lysates. The minor band detected could be the glycosylated form of sVEGFR1.

### Example 2

### Derivation of Cells from Postpartum Tissue

This example describes the preparation of postpartum-derived cells from placental and umbilical cord tissues. Postpartum umbilical cords and placentae were obtained upon birth of either a full term or pre-term pregnancy. Cells were harvested from five separate donors of umbilicus and placental tissue. Different methods of cell isolation were tested for their ability to yield cells with: 1) the potential to differentiate into cells with different phenotypes; or 2) the potential to provide trophic factors useful for other cells and tissues.

Methods & Materials

Umbilical Cell Isolation:

Umbilical cords were obtained from National Disease Research Interchange (NDR1, Philadelphia, Pa.). The tissues were obtained following normal deliveries. The cell isolation protocol was performed aseptically in a laminar flow hood. To remove blood and debris, the cord was washed in phosphate buffered saline (PBS; Invitrogen, Carlsbad, Calif.) in the presence of antimycotic and antibiotic (100 units/milliliter penicillin, 100 micrograms/milliliter streptomycin, 0.25 micrograms/milliliter amphotericin B). The tissues were then mechanically dissociated in 150 cm² tissue culture plates in the presence of 50 milliliters of medium (DMEM-Low glucose or DMEM-High glucose; Invitrogen), until the tissue was minced into a fine pulp. The chopped tissues were transferred to 50 milliliter conical tubes (approximately 5 grams of tissue per tube).

The tissue was then digested in either DMEM-Low glucose medium or DMEM-High glucose medium, each containing antimycotic and antibiotic as described above. In some experiments, an enzyme mixture of collagenase and dispase was used (“C:D”) collagenase (Sigma, St Louis, Mo.), 500 Units/milliliter; and dispase (Invitrogen), 50 Units/milliliter in DMEM-Low glucose medium). In other experiments a mixture of collagenase, dispase and hyaluronidase (“C:D:H”) was used (collagenase, 500 Units/milliliter; dispase, 50 Units/milliliter; and hyaluronidase (Sigma), 5 Units/milliliter, in DMEM-Low glucose). The conical tubes containing the tissue, medium and digestion enzymes were incubated at 37° C. in an orbital shaker (Environ, Brooklyn, N.Y.) at 225 rpm for 2 hrs.

After digestion, the tissues were centrifuged at 150×g for 5 minutes, and the supernatant was aspirated. The pellet was resuspended in 20 milliliters of Growth Medium (DMEM-Low glucose (Invitrogen), 15 percent (v/v) fetal bovine serum (FBS; defined bovine serum; Lot#AND18475; Hyclone, Logan, Utah), 0.001% (v/v) 2-mercaptoethanol (Sigma), 1 milliliter per 100 milliliters of antibiotic/antimycotic as described above. The cell suspension was filtered through a 70-micrometer nylon cell strainer (BD Biosciences). An additional 5 milliliters rinse comprising Growth Medium was passed through the strainer. The cell suspension was then passed through a 40-micrometer nylon cell strainer (BD Biosciences) and chased with a rinse of an additional 5 milliliters of Growth Medium.

The filtrate was resuspended in Growth Medium (total volume 50 milliliters) and centrifuged at 150×g for 5 minutes. The supernatant was aspirated and the cells were resuspended in 50 milliliters of fresh Growth Medium. This process was repeated twice more.

Upon the final centrifugation, supernatant was aspirated and the cell pellet was resuspended in 5 milliliters of fresh Growth Medium. The number of viable cells was determined using Trypan Blue staining. Cells were then cultured under standard conditions.

The cells isolated from umbilical cords were seeded at 5,000 cells/cm² onto gelatin-coated T-75 cm² flasks (Corning Inc., Corning, N.Y.) in Growth Medium with antibiotics/antimycotics as described above. After 2 days (in various experiments, cells were incubated from 2-4 days), spent medium was aspirated from the flasks. Cells were washed with PBS three times to remove debris and blood-derived cells. Cells were then replenished with Growth Medium and allowed to grow to confluence (about 10 days from passage 0) to passage 1. On subsequent passages (from passage 1 to 2 and so on), cells reached sub-confluence (75-85 percent confluence) in 4-5 days. For these subsequent passages, cells were seeded at 5000 cells/cm². Cells were grown in a humidified incubator with 5 percent carbon dioxide and atmospheric oxygen, at 37° C.

Placental Cell Isolation:

Placental tissue was obtained from NDRI (Philadelphia, Pa.). The tissues were from a pregnancy and were obtained at the time of a normal surgical delivery. Placental cells were isolated as described for umbilical cell isolation.

The following example applies to the isolation of separate populations of maternal-derived and neonatal-derived cells from placental tissue.

The cell isolation protocol was performed aseptically in a laminar flow hood. The placental tissue was washed in phosphate buffered saline (PBS; Invitrogen, Carlsbad, Calif.) in the presence of antimycotic and antibiotic (as described above) to remove blood and debris. The placental tissue was then dissected into three sections: top-line (neonatal side or aspect), mid-line (mixed cell isolation neonatal and maternal) and bottom line (maternal side or aspect).

The separated sections were individually washed several times in PBS with antibiotic/antimycotic to further remove blood and debris. Each section was then mechanically dissociated in 150 cm² tissue culture plates in the presence of 50 milliliters of DMEM-Low glucose, to a fine pulp. The pulp was transferred to 50 milliliter conical tubes. Each tube contained approximately 5 grams of tissue. The tissue was digested in either DMEM-Low glucose or DMEM-High glucose medium containing antimycotic and antibiotic (100 U/milliliter penicillin, 100 micrograms/milliliter streptomycin, 0.25 micrograms/milliliter amphotericin B) and digestion enzymes. In some experiments an enzyme mixture of collagenase and dispase (“C:D”) was used containing collagenase (Sigma, St Louis, Mo.) at 500 Units/milliliter and dispase (Invitrogen) at 50 Units/milliliter in DMEM-Low glucose medium. In other experiments a mixture of collagenase, dispase and hyaluronidase (C:D:H) was used (collagenase, 500 Units/milliliter; dispase, 50 Units/milliliter; and hyaluronidase (Sigma), 5 Units/milliliter in DMEM-Low glucose). The conical tubes containing the tissue, medium, and digestion enzymes were incubated for 2 h at 37° C. in an orbital shaker (Environ, Brooklyn, N.Y.) at 225 rpm.

After digestion, the tissues were centrifuged at 150^{x}g for 5 minutes, the resultant supernatant was aspirated off. The pellet was resuspended in 20 milliliters of Growth Medium with penicillin/streptomycin/amphotericin B. The cell suspension was filtered through a 70 micometer nylon cell strainer (BD Biosciences), chased by a rinse with an additional 5 milliliters of Growth Medium. The total cell suspension was passed through a 40 micometer nylon cell strainer (BD Biosciences) followed with an additional 5 milliliters of Growth Medium as a rinse.

The filtrate was resuspended in Growth Medium (total volume 50 milliliters) and centrifuged at 150×g for 5 minutes. The supernatant was aspirated and the cell pellet was resuspended in 50 milliliters of fresh Growth Medium. This process was repeated twice more. After the final centrifugation, supernatant was aspirated and the cell pellet was resuspended in 5 milliliters of fresh Growth Medium. A cell count was determined using the Trypan Blue Exclusion test. Cells were then cultured at standard conditions.

LIBERASE Cell Isolation:

Cells were isolated from umbilicus tissues in DMEM-Low glucose medium with LIBERASE (Boehringer Mannheim Corp., Indianapolis, Ind.) (2.5 milligrams per milliliter, Blendzyme 3; Roche Applied Sciences, Indianapolis, Ind.) and hyaluronidase (5 Units/milliliter, Sigma). Digestion of the tissue and isolation of the cells was as described for other protease digestions above, using the LIBERASE/hyaluronidase mixture in place of the C:D or C:D:H enzyme mixture. Tissue digestion with LIBERASE resulted in the isolation of cell populations from postpartum tissues that expanded readily.

Cell Isolation Using Other Enzyme Combinations:

Procedures were compared for isolating cells from the umbilical cord using differing enzyme combinations. Enzymes compared for digestion included: i) collagenase; ii) dispase; iii) hyaluronidase; iv) collagenase: dispase mixture (C:D); v) collagenase: hyaluronidase mixture (C:H); vi) dispase: hyaluronidase mixture (D:H); and vii) collagenase: dispase: hyaluronidase mixture (C:D:H). Differences in cell isolation utilizing these different enzyme digestion conditions were observed (Table 2-1).

Results

Cell Isolation Using Different Enzyme Combinations:

The combination of C:D:H, provided the best cell yield following isolation, and generated cells, which expanded for many more generations in culture than the other conditions (Table 2-1). An expandable cell population was not attained using collagenase or hyaluronidase alone. No attempt was made to determine if this result is specific to the collagen that was tested.

TABLE 2-1
Isolation of cells from umbilical cord
tissue using varying enzyme combinations

| | Cells | | Cell |
|---|---|---|---|
| Enzyme Digest | Isolated | | Expansion |
| Collagenase | X | | X |
| Dispase | + | (>10 h) | + |
| Hyaluronidase | X | | X |
| Collagenase:Dispase | ++ | (<3 h) | ++ |
| Collagenase:Hyaluronidase | ++ | (<3 h) | + |
| Dispase:Hyaluronidase | + | (>10 h) | + |
| Collagenase:Dispase:Hyaluronidase | +++ | (<3 h) | +++ |

Key: + = good, ++ = very good, +++ = excellent, X = no success

Isolation of Cells Using Different Enzyme Combinations and Growth Conditions:

Cells attached and expanded well between passage 0 and 1 under all conditions tested for enzyme digestion and growth (Table 2-2). Cells in experimental conditions 5-8 and 13-16 proliferated well up to 4 passages after seeding, at which point they were cryopreserved. All cells were cryopreserved for further investigation.

TABLE 2-2
Isolation and culture expansion of postpartum cells under varying conditions:

| Condition | Medium | 15% FBS | BME | Gelatin | 20% O₂ | Growth Factors |
|---|---|---|---|---|---|---|
| 1 | DMEM-Lg | Y | Y | Y | Y | N |
| 2 | DMEM-Lg | Y | Y | Y | N (5%) | N |
| 3 | DMEM-Lg | Y | Y | N | Y | N |
| 4 | DMEM-Lg | Y | Y | N | N (5%) | N |
| 5 | DMEM-Lg | N (2%) | Y | N (Laminin) | Y | EGF/FGF (20 ng/ml) |
| 6 | DMEM-Lg | N (2%) | Y | N (Laminin) | N (5%) | EGF/FGF (20 ng/ml) |
| 7 | DMEM-Lg | N (2%) | Y | N (Fibronectin) | Y | PDGF/VEGF |
| 8 | DMEM-Lg | N (2%) | Y | N (Fibronectin) | N (5%) | PDGF/VEGF |
| 9 | DMEM-Lg | Y | N | Y | Y | N |
| 10 | DMEM-Lg | Y | N | Y | N (5%) | N |
| 11 | DMEM-Lg | Y | N | N | Y | N |
| 12 | DMEM-Lg | Y | N | N | N (5%) | N |
| 13 | DMEM-Lg | N (2%) | N | N (Laminin) | Y | EGF/FGF (20 ng/ml) |
| 14 | DMEM-Lg | N (2%) | N | N (Laminin) | N (5%) | EGF/FGF (20 ng/ml) |
| 15 | DMEM-Lg | N (2%) | N | N (Fibronectin) | Y | PDGF/VEGF |
| 16 | DMEM-Lg | N (2%) | N | N (Fibronectin) | N (5%) | PDGF/VEGF |

Summary:

Populations of cells can be derived from umbilical cord and placental tissue efficiently using the enzyme combination collagenase (a matrix metalloprotease), dispase (a neutral protease) and hyaluronidase (a mucolytic enzyme that breaks down hyaluronic acid). LIBERASE, which is a Blendzyme, may also be used. Specifically, Blendzyme 3, which is collagenase (4 Wunsch units/g) and thermolysin (1714 casein Units/g) was also used together with hyaluronidase to isolate cells. These cells expanded readily over many passages when cultured in Growth Medium on gelatin-coated plastic.

### Example 3

### Karyotype Analysis of Postpartum-Derived Cells

Cell lines used in cell therapy are preferably homogeneous and free from any contaminating cell type. Cells used in cell therapy should have a normal chromosome number (46) and structure. To identify placenta- and umbilicus-derived cell lines that are homogeneous and free from cells of non-postpartum tissue origin, karyotypes of cell samples were analyzed.

Methods & Materials

PPDCs from postpartum tissue of a male neonate were cultured in Growth Medium containing penicillin/streptomycin. Postpartum tissue from a male neonate (X,Y) was selected to allow distinction between neonatal-derived cells and maternal derived cells (X,X). Cells were seeded at 5,000 cells per square centimeter in Growth Medium in a T25 flask (Corning Inc., Corning, N.Y.) and expanded to 80% confluence. A T25 flask containing cells was filled to the neck with Growth Medium. Samples were delivered to a clinical cytogenetics laboratory by courier (estimated lab to lab transport time is one hour). Cells were analyzed during metaphase when the chromosomes are best visualized. Of twenty cells in metaphase counted, five were analyzed for normal homogeneous karyotype number (two). A cell sample was characterized as homogeneous if two karyotypes were observed. A cell sample was characterized as heterogeneous if more than two karyotypes were observed. Additional metaphase cells were counted and analyzed when a heterogeneous karyotype number (four) was identified.

Results

All cell samples sent for chromosome analysis were interpreted as exhibiting a normal appearance. Three of the 16 cell lines analyzed exhibited a heterogeneous phenotype (XX and XY) indicating the presence of cells derived from both neonatal and maternal origins (Table 3-1). Cells derived from tissue Placenta-N were isolated from the neonatal aspect of placenta. At passage zero, this cell line appeared homogeneous XY. However, at passage nine, the cell line was heterogeneous (XX/XY), indicating a previously undetected presence of cells of maternal origin.

TABLE 3-1
Karyotype results of PPDCs.

| | | Metaphase | Metaphase | Number | |
|---|---|---|---|---|---|
| | | cells | cells | of | ISCN |
| Tissue | passage | counted | analyzed | karyotypes | Karyotype |
| Placenta | 22 | 20 | 5 | 2 | 46, XX |
| Umbilical | 23 | 20 | 5 | 2 | 46, XX |
| Umbilical | 6 | 20 | 5 | 2 | 46, XY |
| Placenta | 2 | 20 | 5 | 2 | 46, XX |
| Umbilical | 3 | 20 | 5 | 2 | 46, XX |
| Placenta-N | 0 | 20 | 5 | 2 | 46, XY |
| Placenta-V | 0 | 20 | 5 | 2 | 46, XY |
| Placenta-M | 0 | 21 | 5 | 4 | 46, XY[18]/ |
| | | | | | 46, XX[3] |
| Placenta-M | 4 | 20 | 5 | 2 | 46, XX |
| Placenta-N | 9 | 25 | 5 | 4 | 46, XY[5]/ |
| | | | | | 46, XX[20] |
| Placenta-N | 1 | 20 | 5 | 2 | 46 XY |
| C1 | | | | | |
| Placenta-N | 1 | 20 | 6 | 4 | 46, XY[2]/ |
| C3 | | | | | 46, XX[18] |
| Placenta-N | 1 | 20 | 5 | 2 | 46, XY |
| C4 | | | | | |
| Placenta-N | 1 | 20 | 5 | 2 | 46, XY |
| C15 | | | | | |
| Placenta-N | 1 | 20 | 5 | 2 | 46, XY |
| C20 | | | | | |
| Placenta-N | 1 | 20 | 5 | 2 | 46, XY |
| C22 | | | | | |

Key: N-Neonatal side; V-villous region; M-maternal side; C-clone

Summary:

Chromosome analysis identified placenta- and umbilicus-derived cells whose karyotypes appeared normal as interpreted by a clinical cytogenetic laboratory. Karyotype analysis also identified cell lines free from maternal cells, as determined by homogeneous karyotype.

### Example 4

### Evaluation of Human Postpartum-Derived Cell Surface Markers by Flow Cytometry

Characterization of cell surface proteins or “markers” by flow cytometry can be used to determine a cell line's identity. The consistency of expression can be determined from multiple donors, and in cells exposed to different processing and culturing conditions. Postpartum-derived cell (PPDC) lines isolated from the placenta and umbilicus were characterized (by flow cytometry), providing a profile for the identification of these cell lines.

Methods & Materials

Media and Culture Vessels:

Cells were cultured in Growth Medium (Gibco Carlsbad, Calif.) with penicillin/streptomycin. Cells were cultured in plasma-treated T75, T150, and T225 tissue culture flasks (Corning Inc., Corning, N.Y.) until confluent. The growth surfaces of the flasks were coated with gelatin by incubating 2% (w/v) gelatin (Sigma, St. Louis, Mo.) for 20 minutes at room temperature.

Antibody Staining and Flow Cytometry Analysis:

Adherent cells in flasks were washed in PBS and detached with Trypsin/EDTA. Cells were harvested, centrifuged, and resuspended in 3% (v/v) FBS in PBS at a cell concentration of 1×10⁷ per milliliter. In accordance to the manufacture's specifications, antibody to the cell surface marker of interest (see below) was added to one hundred microliters of cell suspension and the mixture was incubated in the dark for 30 minutes at 4° C. After incubation, cells were washed with PBS and centrifuged to remove unbound antibody. Cells were resuspended in 500 microliter PBS and analyzed by flow cytometry. Flow cytometry analysis was performed with a FACScalibur™ instrument (Becton Dickinson, San Jose, Calif.). Table 4-1 lists the antibodies to cell surface markers that were used.

TABLE 4-1
Antibodies used in characterizing cell surface markers.

| | | Catalog |
|---|---|---|
| Antibody | Manufacture | Number |
| CD10 | BD Pharmingen (San Diego, CA) | 555375 |
| CD13 | BD Pharmingen (San Diego, CA) | 555394 |
| CD31 | BD Pharmingen (San Diego, CA) | 555446 |
| CD34 | BD Pharmingen (San Diego, CA) | 555821 |
| CD44 | BD Pharmingen (San Diego, CA) | 555478 |
| CD45RA | BD Pharmingen (San Diego, CA) | 555489 |
| CD73 | BD Pharmingen (San Diego, CA) | 550257 |
| CD90 | BD Pharmingen (San Diego, CA) | 555596 |
| CD117 | BD Biosciences (San Jose, CA) | 340529 |
| CD141 | BD Pharmingen (San Diego, CA) | 559781 |
| PDGFr-alpha | BD Pharmingen (San Diego, CA) | 556002 |
| HLA-A, B, C | BD Pharmingen (San Diego, CA) | 555553 |
| HLA-DR, DP, DQ | BD Pharmingen (San Diego, CA) | 555558 |
| IgG-FITC | Sigma (St. Louis, MO) | F-6522 |
| IgG-PE | Sigma (St. Louis, MO) | P-4685 |

Placenta and Umbilicus Comparison:

Placenta-derived cells were compared to umbilicus-derive cells at passage 8.

Passage to Passage Comparison:

Placenta- and umbilicus-derived cells were analyzed at passages 8, 15, and 20.

Donor to Donor Comparison:

To compare differences among donors, placenta-derived cells from different donors were compared to each other, and umbilicus-derived cells from different donors were compared to each other.

Surface Coating Comparison:

Placenta-derived cells cultured on gelatin-coated flasks was compared to placenta-derived cells cultured on uncoated flasks. Umbilicus-derived cells cultured on gelatin-coated flasks was compared to umbilicus-derived cells cultured on uncoated flasks.

Digestion Enzyme Comparison:

Four treatments used for isolation and preparation of cells were compared. Cells isolated from placenta by treatment with 1) collagenase; 2) collagenase/dispase; 3) collagenase/hyaluronidase; and 4) collagenase/hyaluronidase/dispase were compared.

Placental Layer Comparison:

Cells derived from the maternal aspect of placental tissue were compared to cells derived from the villous region of placental tissue and cells derived from the neonatal fetal aspect of placenta.

Results

Placenta Vs. Umbilicus Comparison:

Placenta- and umbilicus-derived cells analyzed by flow cytometry showed positive expression of CD10, CD13, CD44, CD73, CD90, PDGFr-alpha and HLA-A, B, C, indicated by the increased values of fluorescence relative to the IgG control. These cells were negative for detectable expression of CD31, CD34, CD45, CD117, CD141, and HLA-DR, DP, DQ, indicated by fluorescence values comparable to the IgG control. Variations in fluorescence values of positive curves were accounted. The mean (i.e. CD13) and range (i.e. CD90) of the positive curves showed some variation, but the curves appeared normal, confirming a homogenous population. Both curves individually exhibited values greater than the IgG control.

Passage to Passage Comparison—Placenta-Derived Cells:

Placenta-derived cells at passages 8, 15, and 20 analyzed by flow cytometry all were positive for expression of CD10, CD13, CD44, CD73, CD90, PDGFr-alpha and HLA-A, B, C, as reflected in the increased value of fluorescence relative to the IgG control. The cells were negative for expression of CD31, CD34, CD45, CD117, CD141, and HLA-DR, DP, DQ having fluorescence values consistent with the IgG control.

Passage to Passage Comparison—Umbilicus-Derived Cells:

Umbilicus-derived cells at passage 8, 15, and 20 analyzed by flow cytometry all expressed CD10, CD13, CD44, CD73, CD90, PDGFr-alpha and HLA-A, B, C, indicated by increased fluorescence relative to the IgG control. These cells were negative for CD31, CD34, CD45, CD117, CD141, and HLA-DR, DP, DQ, indicated by fluorescence values consistent with the IgG control.

Donor to Donor Comparison—Placenta-Derived Cells:

Placenta-derived cells isolated from separate donors analyzed by flow cytometry each expressed CD10, CD13, CD44, CD73, CD90, PDGFr-alpha and HLA-A, B, C, with increased values of fluorescence relative to the IgG control. The cells were negative for expression of CD31, CD34, CD45, CD117, CD141, and HLA-DR, DP, DQ as indicated by fluorescence value consistent with the IgG control.

Donor to Donor Comparison—Umbilicus Derived Cells:

Umbilicus-derived cells isolated from separate donors analyzed by flow cytometry each showed positive expression of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ with fluorescence values consistent with the IgG control.

The Effect of Surface Coating with Gelatin on Placenta-Derived Cells:

Placenta-derived cells expanded on either gelatin-coated or uncoated flasks analyzed by flow cytometry all expressed of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ indicated by fluorescence values consistent with the IgG control.

The Effect of Surface Coating with Gelatin on Umbilicus-Derived Cells:

Umbilicus-derived cells expanded on gelatin and uncoated flasks analyzed by flow cytometry all were positive for expression of CD10, CD13, CD44, CD73, CD90, PDGFr-alpha and HLA-A, B, C, with increased values of fluorescence relative to the IgG control. These cells were negative for expression of CD31, CD34, CD45, CD117, CD141, and HLA-DR, DP, DQ, with fluorescence values consistent with the IgG control.

Effect of Enzyme Digestion Procedure Used for Preparation of the Cells on the Cell Surface Marker Profile:

Placenta-derived cells isolated using various digestion enzymes analyzed by flow cytometry all expressed CD10, CD13, CD44, CD73, CD90, PDGFr-alpha and HLA-A, B, C, as indicated by the increased values of fluorescence relative to the IgG control. These cells were negative for expression of CD31, CD34, CD45, CD117, CD141, and HLADR, DP, DQ as indicated by fluorescence values consistent with the IgG control.

Placental Layer Comparison:

Cells isolated from the maternal, villous, and neonatal layers of the placenta, respectively, analyzed by flow cytometry showed positive expression of CD10, CD13, CD44, CD73, CD90, PDGFr-alpha and HLA-A, B, C, as indicated by the increased value of fluorescence relative to the IgG control. These cells were negative for expression of CD31, CD34, CD45, CD117, CD141, and HLA-DR, DP, DQ as indicated by fluorescence values consistent with the IgG control.

Summary:

Analysis of placenta- and umbilicus-derived cells by flow cytometry has established of an identity of these cell lines. Placenta- and umbilicus-derived cells are positive for CD10, CD13, CD44, CD73, CD90, PDGFr-alpha, HLA-A,B,C and negative for CD31, CD34, CD45, CD117, CD141 and HLA-DR, DP, DQ. This identity was consistent between variations in variables including the donor, passage, culture vessel surface coating, digestion enzymes, and placental layer. Some variation in individual fluorescence value histogram curve means and ranges was observed, but all positive curves under all conditions tested were normal and expressed fluorescence values greater than the IgG control, thus confirming that the cells comprise a homogenous population that has positive expression of the markers.

### Example 5

### Immunohistochemical Characterization of Postpartum Tissue Phenotypes

The phenotypes of cells found within human postpartum tissues, namely umbilical cord and placenta, was analyzed by immunohistochemistry.

Methods & Materials

Tissue Preparation:

Human umbilical cord and placenta tissue was harvested and immersion fixed in 4% (w/v) paraformaldehyde overnight at 4° C. Immunohistochemistry was performed using antibodies directed against the following epitopes: vimentin (1:500; Sigma, St. Louis, Mo.), desmin (1:150, raised against rabbit; Sigma; or 1:300, raised against mouse; Chemic on, Temecula, Calif.), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested: antihuman GROalpha-PE (1:100; Becton Dickinson, Franklin Lakes, N.J), antihuman GCP-2 (1:100; Santa Cruz Biotech, Santa Cruz, Calif.), anti-human oxidized LDL receptor 1 (ox-LDL R1; 1:100; Santa Cruz Biotech), and anti-human NOGO-A (1:100; Santa Cruz Biotech). Fixed specimens were trimmed with a scalpel and placed within OCT embedding compound (Tissue-Tek OCT; Sakura, Torrance, Calif.) on a dry ice bath containing ethanol. Frozen blocks were then sectioned (10 μm thick) using a standard cryostat (Leica Microsystems) and mounted onto glass slides for staining.

Immunohistochemistry:

Immunohistochemistry was performed similar to previous studies (e.g., Messina, et al., 2003, Exper. Neurol. 184: 816-829). Tissue sections were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 1 hour to access intracellular antigens. In instances where the epitope of interest would be located on the cell surface (CD34, ox-LDL R1), Triton was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout the procedure. Primary antibodies, diluted in blocking solution, were then applied to the sections for a period of 4 hours at room temperature. Primary antibody solutions were removed, and cultures washed with PBS prior to application of secondary antibody solutions (1 hour at room temperature) containing block along with goat anti-mouse IgG—Texas Red (1:250; Molecular Probes, Eugene, Oreg.) and/or goat anti-rabbit IgG—Alexa 488 (1:250; Molecular Probes) or donkey anti-goat IgG-FITC (1:150; Santa Cruz Biotech). Cultures were washed, and 10 micromolar DAPI (Molecular Probes) was applied for 10 minutes to visualize cell nuclei.

Following immunostaining, fluorescence was visualized using the appropriate fluorescence filter on an Olympus inverted epi-fluorescent microscope (Olympus, Melville, N.Y.). Positive staining was represented by fluorescence signal above control staining. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

Results

Umbilical Cord Characterization:

Vimentin, desmin, SMA, CKI8, vWF, and CD34 markers were expressed in a subset of the cells found within umbilical cord. In particular, vWF and CD34 expression were restricted to blood vessels contained within the cord. CD34+ cells were on the innermost layer (lumen side). Vimentin expression was found throughout the matrix and blood vessels of the cord. SMA was limited to the matrix and outer walls of the artery & vein, but not contained with the vessels themselves. CK18 and desmin were observed within the vessels only, desmin being restricted to the middle and outer layers.

Placenta Characterization:

Vimentin, desmin, SMA, CKI8, vWF, and CD34 were all observed within the placenta and regionally specific.

GROalpha, GCP-2, Ox-LDL RI, and NOGO-A Tissue Expression:

None of these markers were observed within umbilical cord or placental tissue.

Summary:

Vimentin, desmin, alpha-smooth muscle actin, cytokeratin 18, von Willebrand Factor, and CD34 are expressed in cells within human umbilical cord and placenta.

### Example 6

### Analysis of Postpartum Tissue-Derived Cells Using Oligonucleotide Arrays

Affymetrix GENECHIP arrays were used to compare gene expression profiles of umbilicus- and placenta-derived cells with fibroblasts, human mesenchymal stem cells, and another cell line derived from human bone marrow. This analysis provided a characterization of the postpartum-derived cells and identified unique molecular markers for these cells.

Methods & Materials

Isolation and Culture of Cells:

Human umbilical cords and placenta were obtained from National Disease Research Interchange (NDRI, Philadelphia, Pa.) from normal full term deliveries with patient consent. The tissues were received and cells were isolated as described in Example 5. Cells were cultured in Growth Medium (using DMEM-LG) on gelatin-coated tissue culture plastic flasks. The cultures were incubated at 37° C. with 5% CO₂.

Human dermal fibroblasts were purchased from Cambrex Incorporated (Walkersville, Md.; Lot number 9F0844) and ATCC CRL-1501 (CCD39SK). Both lines were cultured in DMEM/F12 medium (Invitrogen, Carlsbad, Calif.) with 10% (v/v) fetal bovine serum (Hyclone) and penicillin/streptomycin (Invitrogen). The cells were grown on standard tissue-treated plastic.

Human mesenchymal stem cells (hMSC) were purchased from Cambrex Incorporated (Walkersville, Md.; Lot numbers 2F1655, 2F1656 and 2F1657) and cultured according to the manufacturer's specifications in MSCGM Media (Cambrex). The cells were grown on standard tissue cultured plastic at 37° C. with 5% CO₂.

Human iliac crest bone marrow was received from the NDRI with patient consent. The marrow was processed according to the method outlined by Ho, et al. (W003/025149). The marrow was mixed with lysis buffer (155 mM NH 4Cl, 10 mM KHCO₃, and 0.1 mM EDTA, pH 7.2) at a ratio of 1 part bone marrow to 20 parts lysis buffer. The cell suspension was vortexed, incubated for 2 minutes at ambient temperature, and centrifuged for 10 minutes at 500^{x}g. The supernatant was discarded and the cell pellet was resuspended in Minimal Essential Medium-alpha (Invitrogen) supplemented with 10% (v/v) fetal bovine serum and 4 mM glutamine. The cells were centrifuged again and the cell pellet was resuspended in fresh medium. The viable mononuclear cells were counted using trypan-blue exclusion (Sigma, St. Louis, Mo.). The mononuclear cells were seeded in tissue-cultured plastic flasks at 5×10⁴ cells/cm². The cells were incubated at 37° C. with 5% CO₂ at either standard atmospheric O₂ or at 5% O₂. Cells were cultured for 5 days without a media change. Media and non-adherent cells were removed after 5 days of culture. The adherent cells were maintained in culture.

Isolation of mRNA and GENECHIP Analysis:

Actively growing cultures of cells were removed from the flasks with a cell scraper in cold PBS. The cells were centrifuged for 5 minutes at 300^{x}g. The supernatant was removed and the cells were resuspended in fresh PBS and centrifuged again. The supernatant was removed and the cell pellet was immediately frozen and stored at −80° C. Cellular mRNA was extracted and transcribed into cDNA, which was then transcribed into cRNA and biotin-labeled. The biotin-labeled cRNA was hybridized with HG-U133A GENECHIP oligonucleotide array (Affymetrix, Santa Clara Calif.). The hybridization and data collection was performed according to the manufacturer's specifications. Analyses were performed using “Significance Analysis of Microarrays” (SAM) version 1.21 computer software (Stanford University; Tusher, V. G. et al., 2001, PNAS USA 98: 5116-5121).\

Results

Fourteen different populations of cells were analyzed. The cells along with passage information, culture substrate, and culture media are listed in Table 6-1.

TABLE 6-1
Cells analyzed by the microarray study. The cells lines
are listed by their identification code along with passage at the
time of analysis, cell growth substrate, and growth media.

| Cell Population | Passage | Substrate | Medium |
|---|---|---|---|
| Umbilical (022803) | 2 | Gelatin | DMEM, 15% FBS, 2-ME |
| Umbilical (042103) | 3 | Gelatin | DMEM, 15% FBS, 2-ME |
| Umbilical (071003) | 4 | Gelatin | DMEM, 15% FBS, 2-ME |
| Placenta (042203) | 12 | Gelatin | DMEM, 15% FBS, 2-ME |
| Placenta (042903) | 4 | Gelatin | DMEM, 15% FBS, 2-ME |
| Placenta (071003) | 3 | Gelatin | DMEM, 15% FBS, 2-ME |
| ICBM (070203) (5% O₂) | 3 | Plastic | MEM 10% FBS |
| ICBM (062703) (std O₂) | 5 | Plastic | MEM 10% FBS |
| ICBM (062703)(5% O₂) | 5 | Plastic | MEM 10% FBS |
| hMSC (Lot 2F1655) | 3 | Plastic | MSCGM |
| hMSC (Lot 2F1656) | 3 | Plastic | MSCGM |
| hMSC (Lot 2F1657) | 3 | Plastic | MSCGM |
| hFibroblast (9F0844) | 9 | Plastic | DMEM-F12, 10% FBS |
| hFibroblast (CCD39SK) | 4 | Plastic | DMEM-F12, 10% FBS |

The data were evaluated by a Principle Component Analysis, analyzing the 290 genes that were differentially expressed in the cells. This analysis allows for a relative comparison for the similarities between the populations.

Table 6-2 shows the Euclidean distances that were calculated for the comparison of the cell pairs. The Euclidean distances were based on the comparison of the cells based on the 290 genes that were differentially expressed among the cell types. The Euclidean distance is inversely proportional to similarity between the expression of the 290 genes (i.e., the greater the distance, the less similarity exists).

TABLE 6-2
The Euclidean Distances for the Cell Pairs.

| Cell Pair | Euclidean Distance |
|---|---|
| ICBM-hMSC | 24.71 |
| Placenta-umbilical | 25.52 |
| ICBM-Fibroblast | 36.44 |
| ICBM-placenta | 37.09 |
| Fibroblast-MSC | 39.63 |
| ICBM-Umbilical | 40.15 |
| Fibroblast-Umbilical | 41.59 |
| MSC-Placenta | 42.84 |
| MSC-Umbilical | 46.86 |
| ICBM-placenta | 48.41 |

Tables 6-3, 6-4, and 6-5 show the expression of genes increased in placenta-derived cells (Table 6-3), increased in umbilicus-derived cells (Table 6-4), and reduced in umbilicus- and placenta-derived cells (Table 6-5). The column entitled “Probe Set ID” refers to the manufacturer's identification code for the sets of several oligonucleotide probes located on a particular site on the chip, which hybridize to the named gene (column “Gene Name”), comprising a sequence that can be found within the NCBI (GenBank) database at the specified accession number (column “NCBI Accession Number”).

TABLE 6-3
Genes shown to have specifically increased expression in the placenta-
derived cells as compared to other cell lines assayed
Genes Increased in Placenta-Derived Cells

| | | NCBI |
|---|---|---|
| Probe | | Accession |
| Set ID | Gene Name | Number |
| 209732_at | C-type (calcium dependent, carbohydrate- | AF070642 |
| | recognition domain) lectin, | |
| | superfamily member 2 (activation-induced) | |
| 206067_s_at | Wilms tumor 1 | NM_024426 |
| 207016_s_at | aldehyde dehydrogenase 1 family, member A2 | AB015228 |
| 206367_at | renin | NM_000537 |
| 210004_at | oxidized low density lipoprotein | AF035776 |
| | (lectin-like) receptor 1 | |
| 214993_at | Homo sapiens, clone IMAGE: 4179671, | AF070642 |
| | mRNA, partial cds | |
| 202178_at | protein kinase C, zeta | NM_002744 |
| 209780_at | hypothetical protein DKFZp564F013 | AL136883 |
| 204135_at | downregulated in ovarian cancer 1 | NM_014890 |
| 213542_at | Homo sapiens mRNA; cDNA DKFZp547K1113 | AI246730 |
| | (from clone DKFZp547K1113) | |

TABLE 6-4
Genes shown to have specifically increased expression in the
umbilicus-derived cells as compared to other cell lines assayed
Genes Increased in Umbilicus-Derived Cells

| | | NCBI |
|---|---|---|
| Probe | | Accession |
| Set ID | Gene Name | Number |
| 202859_x_at | interleukin 8 | NM_000584 |
| 211506_s_at | interleukin 8 | AF043337 |
| 210222_s_at | reticulon 1 | BC000314 |
| 204470_at | chemokine (C-X-C motif) ligand 1 | NM_001511 |
| | (melanoma growth stimulating activity | |
| 206336_at | chemokine (C-X-C motif) ligand 6 | NM_002993 |
| | (granulocyte chemotactic protein 2) | |
| 207850_at | chemokine (C-X-C motif) ligand 3 | NM_002090 |
| 203485_at | reticulon 1 | NM_021136 |
| 202644_s_at | tumor necrosis factor, alpha- | NM_006290 |
| | induced protein 3 | |

TABLE 6-5
Genes shown to have decreased expression in umbilicus- and placenta-
derived cells as compared to other cell lines assayed
Genes Decreased in Umbilicus- and Placenta-Derived Cells

| | | NCBI |
|---|---|---|
| Probe | | Accession |
| Set ID | Gene name | Number |
| 210135_s_at | short stature homeobox 2 | AF022654.1 |
| 205824_at | heat shock 27 kDa protein 2 | NM_001541.1 |
| 209687_at | chemokine (C-X-C motif) ligand 12 | U19495.1 |
| | (stromal cell-derived factor 1) | |
| 203666_at | chemokine (C-X-C motif) ligand 12 | NM_000609.1 |
| | (stromal cell-derived factor 1) | |
| 212670_at | elastin (supravalvular aortic stenosis, | AA479278 |
| | Williams-Beuren syndrome) | |
| 213381_at | Homo sapiens mRNA; cDNA | N91149 |
| | DKFZp586M2022 (from clone | |
| | DKFZp586M2022) | |
| 206201_s_at | mesenchyme homeo box 2 | NM_005924.1 |
| | (growth arrest-specific homeo box) | |
| 205817_at | sine oculis homeobox homolog | NM_005982.1 |
| | 1 (Drosophila) | |
| 209283_at | crystallin, alpha B | AF007162.1 |
| 212793_at | dishevelled associated activator | BF513244 |
| | of morphogenesis 2 | |
| 213488_at | DKFZP58662420 protein | AL050143.1 |
| 209763_at | similar to neuralin 1 | AL049176 |
| 205200_at | tetranectin (plasminogen binding protein) | NM_003278.1 |
| 205743_at | src homology three (SH3) and | NM_003149.1 |
| | cysteine rich domain | |
| 200921_s_at | B-cell translocation gene 1, | NM_001731.1 |
| | anti-proliferative | |
| 206932_at | cholesterol 25-hydroxylase | NM_003956.1 |
| 204198_s_at | runt-related transcription factor 3 | AA541630 |
| 219747_at | hypothetical protein FLJ23191 | NM_024574.1 |
| 204773_at | interleukin 11 receptor, alpha | NM_004512.1 |
| 202465_at | procollagen C-endopeptidase enhancer | NM_002593.2 |
| 203706_s_at | frizzled homolog 7 (Drosophila) | NM_003507.1 |
| 212736_at | hypothetical gene BC008967 | BE299456 |
| 214587_at | collagen, type VIII, alpha 1 | BE877796 |
| 201645_at | tenascin C (hexabrachion) | NM_002160.1 |
| 210239_at | iroquois homeobox protein 5 | U90304.1 |
| 203903_s_at | Hephaestin | NM_014799.1 |
| 205816_at | integrin, beta 8 | NM_002214.1 |
| 203069_at | synaptic vesicle glycoprotein 2 | NM_014849.1 |
| 213909_at | Homo sapiens cDNA FLJ12280 fis, | AU147799 |
| | clone MAMMA1001744 | |
| 206315_at | cytokine receptor-like factor 1 | NM_004750.1 |
| 204401_at | potassium intermediate/small conductance | NM_002250.1 |
| | calcium-activated channel, | |
| | subfamily N, member 4 | |
| 216331_at | integrin, alpha 7 | AK022548.1 |
| 209663_s_at | integrin, alpha 7 | AF072132.1 |
| 213125_at | DKFZP586L151 protein | AW007573 |
| 202133_at | transcriptional co-activator with | AA081084 |
| | PDZ-binding motif (TAZ) | |
| 206511_s_at | sine oculis homeobox homolog | NM_016932.1 |
| | 2 (Drosophila) | |
| 213435_at | KIAA1034 protein | AB028957.1 |
| 206115_at | early growth response 3 | NM_004430.1 |
| 213707_s_at | distal-less homeo box 5 | NM_005221.3 |
| 218181_s_at | hypothetical protein FLJ20373 | NM_017792.1 |
| 209160_at | aldo-keto reductase family 1, | AB018580.1 |
| | member C3 (3-alpha hydroxysteroid | |
| | dehydrogenase, type II) | |
| 213905_x_at | Biglycan | AA845258 |
| 201261_x_at | Biglycan | BC002416.1 |
| 202132_at | transcriptional co-activator with | AA081084 |
| | PDZ-binding motif (TAZ) | |
| 214701_s_at | fibronectin 1 | AJ276395.1 |
| 213791_at | Proenkephalin | NM_006211.1 |
| 205422_s_at | integrin, beta-like 1 | NM_004791.1 |
| | (with EGF-like repeat domains) | |
| 214927_at | Homo sapiens mRNA full length insert | AL359052.1 |
| | cDNA clone EUROIMAGE 1968422 | |
| 206070_s_at | EphA3 | AF213459.1 |
| 212805_at | KIAA0367 protein | AB002365.1 |
| 219789_at | natriuretic peptide receptor C/ | AI628360 |
| | guanylate cyclase C | |
| | (atrionatriuretic peptide receptor C) | |
| 219054_at | hypothetical protein FLJ14054 | NM_024563.1 |
| 213429_at | Homo sapiens mRNA; cDNA | AW025579 |
| | DKFZp5646222 | |
| | (from clone DKFZp5646222) | |
| 204929_s_at | vesicle-associated membrane | NM_006634.1 |
| | protein 5 (myobrevin) | |
| 201843_s_at | EGF-containing fibulin-like | NM_004105.2 |
| | extracellular matrix protein 1 | |
| 221478_at | BCL2/adenovirus E1B 19 kDa | AL132665.1 |
| | interacting protein 3-like | |
| 201792_at | AE binding protein 1 | NM_001129.2 |
| 204570_at | cytochrome c oxidase subunit | NM_001864.1 |
| | VIIa polypeptide 1 (muscle) | |
| 201621_at | neuroblastoma, suppression | NM_005380.1 |
| | of tumorigenicity 1 | |
| 202718_at | insulin-like growth factor | NM_000597.1 |
| | binding protein 2, 36 kDa | |

Tables 6-6, 6-7, and 6-8 show the expression of genes increased in human fibroblasts (Table 6-6), ICBM cells (Table 6-7), and MSCs (Table 6-8).

TABLE 6-6
Genes that were shown to have increased expression in
fibroblasts as compared to the other cell lines assayed.
Genes increased in fibroblasts
dual specificity phosphatase 2
KIAA0527 protein
Homo sapiens cDNA: FLJ23224 fis, clone ADSU02206
dynein, cytoplasmic, intermediate polypeptide 1
ankyrin 3, node of Ranvier (ankyrin G)
inhibin, beta A (activin A, activin AB alpha polypeptide)
ectonucleotide pyrophosphatase/phosphodiesterase 4 (putative function)
KIAA1053 protein
microtubule-associated protein 1A
zinc finger protein 41
HSPC019 protein
Homo sapiens cDNA: FLJ23564 fis, clone LNG10773
Homo sapiens mRNA; cDNA DKFZp564A072 (from clone
DKFZp564A072)
LIM protein (similar to rat protein kinase C-binding enigma)
inhibitor of kappa light polypeptide gene enhancer in B-cells, kinase
complex-associated protein
hypothetical protein FLJ22004
Human (clone CTG-A4) mRNA sequence
ESTs, Moderately similar to cytokine receptor-like factor 2; cytokine
receptor CRL2 precursor [Homo sapiens]
transforming growth factor, beta 2
hypothetical protein MGC29643
antigen identified by monoclonal antibody MRC OX-2
putative X-linked retinopathy protein

TABLE 6-7
Genes that were shown to have increased expression in the ICBM-
derived cells as compared to the other cell lines assayed.
Genes Increased In ICBM Cells
cardiac ankyrin repeat protein
MHC class I region ORF
integrin, alpha 10
hypothetical protein FLJ22362
UDP-N-acetyl-alpha-D-galactosamine:polypeptide
N-acetylgalactosaminyltransferase 3 (GalNAc-T3)
interferon-induced protein 44
SRY (sex determining region Y)-box 9 (campomelic
dysplasia, autosomal sex-reversal)
keratin associated protein 1-1
hippocalcin-like 1
jagged 1 (Alagille syndrome)
proteoglycan 1, secretory granule

TABLE 6-8
Genes that were shown to have increased expression in the
MSC cells as compared to the other cell lines assayed.
Genes Increased In MSC Cells
interleukin 26
maltase-glucoamylase (alpha-glucosidase)
nuclear receptor subfamily 4, group A, member 2
v-fos FBJ murine osteosarcoma viral oncogene homolog
hypothetical protein DC42
nuclear receptor subfamily 4, group A, member 2
FBJ murine osteosarcoma viral oncogene homolog B
WNT1 inducible signaling pathway protein 1
MCF.2 cell line derived transforming sequence
potassium channel, subfamily K, member 15
cartilage paired-class homeoprotein 1
Homo sapiens cDNA FLJ12232 fis, clone MAMMA1001206
Homo sapiens cDNA FLJ34668 fis, clone LIVER2000775
jun B proto-oncogene
B-cell CLL/lymphoma 6 (zinc finger protein 51)
zinc finger protein 36, C3H type, homolog (mouse)

Summary:

The present examination was performed to provide a molecular characterization of the postpartum cells derived from umbilical cord and placenta. This analysis included cells derived from three different umbilical cords and three different placentas. The examination also included two different lines of dermal fibroblasts, three lines of mesenchymal stem cells, and three lines of iliac crest bone marrow cells. The mRNA that was expressed by these cells was analyzed using an oligonucleotide array that contained probes for 22,000 genes. Results showed that 290 genes are differentially expressed in these five different cell types. These genes include ten genes that are specifically increased in the placenta-derived cells and seven genes specifically increased in the umbilical cord-derived cells. Fifty-four genes were found to have specifically lower expression levels in placenta and umbilical cord, as compared with the other cell types. The expression of selected genes has been confirmed by PCR (see the example that follows). These results demonstrate that the postpartum-derived cells have a distinct gene expression profile, for example, as compared to bone marrow-derived cells and fibroblasts.

### Example 7

### Cell Markers in Postpartum-Derived Cells

In the preceding example, similarities and differences in cells derived from the human placenta and the human umbilical cord were assessed by comparing their gene expression profiles with those of cells derived from other sources (using an oligonucleotide array). Six “signature” genes were identified: oxidized LDL receptor 1, interleukin-8, rennin, reticulon, chemokine receptor ligand 3 (CXC ligand 3), and granulocyte chemotactic protein 2 (GCP-2). These “signature” genes were expressed at relatively high levels in postpartum-derived cells.

The procedures described in this example were conducted to verify the microarray data and find concordance/discordance between gene and protein expression, as well as to establish a series of reliable assay for detection of unique identifiers for placenta- and umbilicus-derived cells.

Methods & Materials

Cells:

Placenta-derived cells (three isolates, including one isolate predominately neonatal as identified by karyotyping analysis), umbilicus-derived cells (four isolates), and Normal Human Dermal Fibroblasts (NHDF; neonatal and adult) grown in Growth Medium with penicillin/streptomycin in a gelatin-coated T75 flask. Mesechymal Stem Cells (MSCS) were grown in Mesenchymal Stem Cell Growth Medium Bullet kit (MSCGM; Cambrex, Walkerville, Md.).

For the IL-8 protocol, cells were thawed from liquid nitrogen and plated in gelatin-coated flasks at 5,000 cells/cm², grown for 48 hours in Growth Medium and then grown for further 8 hours in 10 milliliters of serum starvation medium [DMEM—low glucose (Gibco, Carlsbad, Calif.), penicillin/streptomycin (Gibco, Carlsbad, Calif.) and 0.1% (w/v) Bovine Serum Albumin (BSA; Sigma, St. Louis, Mo.)]. After this treatment RNA was extracted and the supernatants were centrifuged at 150×g for 5 minutes to remove cellular debris. Supernatants were then frozen at −80° C. for ELISA analysis.

Cell Culture for ELISA Assay:

Postpartum cells derived from placenta and umbilicus, as well as human fibroblasts derived from human neonatal foreskin were cultured in Growth Medium in gelatin-coated T75 flasks. Cells were frozen at passage 11 in liquid nitrogen. Cells were thawed and transferred to 15-milliliter centrifuge tubes. After centrifugation at 150×g for 5 minutes, the supernatant was discarded. Cells were resuspended in 4 milliliters culture medium and counted. Cells were grown in a 75 cm² flask containing 15 milliliters of Growth Medium at 375,000 cells/flask for 24 hours. The medium was changed to a serum starvation medium for 8 hours. Serum starvation medium was collected at the end of incubation, centrifuged at 14,000^{x}g for 5 minutes (and stored at −20° C.).

To estimate the number of cells in each flask, 2 milliliters of tyrpsin/EDTA (Gibco, Carlsbad, Calif.) was added each flask. After cells detached from the flask, trypsin activity was neutralized with 8 milliliters of Growth Medium. Cells were transferred to a 15 milliliters centrifuge tube and centrifuged at 150×g for 5 minutes. Supernatant was removed and 1 milliliter Growth Medium was added to each tube to resuspend the cells. Cell number was estimated using a hemocytometer.

ELISA Assay:

The amount of IL-8 secreted by the cells into serum starvation medium was analyzed using ELISA assays (R&D Systems, Minneapolis, Minn.). All assays were tested according to the instructions provided by the manufacturer.

Total RNA Isolation:

RNA was extracted from confluent postpartum-derived cells and fibroblasts or for IL-8 expression from cells treated as described above. Cells were lysed with 350 microliters buffer RLT containing beta-mercaptoethanol (Sigma, St. Louis, Mo.) according to the manufacturer's instructions (RNeasy® Mini Kit; Qiagen, Valencia, Calif.). RNA was extracted according to the manufacturer's instructions (RNeasy® Mini Kit; Qiagen, Valencia, Calif.) and subjected to DNase treatment (2.7 U/sample) (Sigma St. Louis, Mo.). RNA was eluted with 50 microliters DEPC-treated water and stored at −80° C.

Reverse Transcription:

RNA was also extracted from human placenta and umbilicus. Tissue (30 milligram) was suspended in 700 microliters of buffer RLT containing 2-mercaptoethanol. Samples were mechanically homogenized and the RNA extraction proceeded according to manufacturer's specification. RNA was extracted with 50 microliters of DEPC-treated water and stored at −80° C. RNA was reversed transcribed using random hexamers with the TaqMan® reverse transcription reagents (Applied Biosystems, Foster City, Calif.) at 25° C. for 10 minutes, 37° C. for 60 minutes, and 95° C. for 10 minutes. Samples were stored at −20° C.

Genes identified by cDNA microarray as uniquely regulated in postpartum cells (signature genes—including oxidized LDL receptor, interleukin-8, rennin and reticulon), were further investigated using real-time and conventional PCR.

Real-Time PCR:

PCR was performed on cDNA samples using Assays-on-Demand® gene expression products: oxidized LDL receptor (Hs00234028); rennin (Hs00166915); reticulon (Hs003825 15); CXC ligand 3 (Hs00171061); GCP-2 (Hs00605742); IL-8 (Hs00174103); and GAPDH (Applied Biosystems, Foster City, Calif.) were mixed with cDNA and TaqMan® Universal PCR master mix according to the manufacturer's instructions (Applied Biosystems, Foster City, Calif.) using a 7000 sequence detection system with ABI Prism 7000 SDS software (Applied Biosystems, Foster City, Calif.). Thermal cycle conditions were initially 50° C. for 2 min and 95° C. for 10 min, followed by 40 cycles of 95° C. for 15 sec and 60° C. for 1 min. PCR data was analyzed according to manufacturer's specifications (User Bulletin #2 from Applied Biosystems for ABI Prism 7700 Sequence Detection System).

Conventional PCR:

Conventional PCR was performed using an ABI PRISM 7700 (Perkin Elmer Applied Biosystems, Boston, Mass., USA) to confirm the results from real-time PCR. PCR was performed using 2 microliters of cDNA solution, lx AmpliTaq Gold universal mix PCR reaction buffer (Applied Biosystems, Foster City, Calif.) and initial denaturation at 94° C. for 5 minutes. Amplification was optimized for each primer set. For IL-8, CXC ligand 3, and reticulon (94° C. for 15 seconds, 55° C. for 15 seconds and 72° C. for 30 seconds for 30 cycles); for rennin (94° C. for 15 seconds, 53° C. for 15 seconds and 72° C. for 30 seconds for 38 cycles); for oxidized LDL receptor and GAPDH (94° C. for 15 seconds, 55° C. for 15 seconds and 72° C. for 30 seconds for 33 cycles). Primers used for amplification are listed in Table 7-1. Primer concentration in the final PCR reaction was 1 micromolar except for GAPDH, which was 0.5 micromolar. GAPDH primers were the same as real-time PCR, except that the manufacturer's TaqMan® probe was not added to the final PCR reaction. Samples were run on 2% (w/v) agarose gel and stained with ethidium bromide (Sigma, St. Louis, Mo.). Images were captured using a 667 Universal Twinpack film (VWR International, South Plainfield, N.J.) using a focal length Polaroid camera (VWR International, South Plainfield, N.J.).

TABLE 7-1
Primers used

| Primer name | Primers |
|---|---|
| Oxidized LDL | S: 5′- GAGAAATCCAAAGAGCAAATGG-3 |
| receptor | (SEQ ID NO: 1) |
| | A: 5′-AGAATGGAAAACTGGAATAGG -3′ |
| | (SEQ ID NO: 2) |
| Renin | S: 5′-TCTTCGATGCTTCGGATTCC -3′ |
| | (SEQ ID NO: 3) |
| | A: 5′-GAATTCTCGGAATCTCTGTTG -3′ |
| | (SEQ ID NO: 4) |
| Reticulon | S: 5′- TTACAAGCAGTGCAGAAAACC-3′ |
| | (SEQ ID NO: 5) |
| | A: 5′- AGTAAACATTGAAACCACAGCC-3′ |
| | (SEQ ID NO: 6) |
| Interleukin-8 | S: 5′- TCTGCAGCTCTGTGTGAAGG-3′ |
| | (SEQ ID NO: 7) |
| | A: 5′-CTTCAAAAACTTCTCCACAACC- 3′ |
| | (SEQ ID NO: 8) |
| Chemokine (CXC) | S: 5′- CCCACGCCACGCTCTCC-3′ |
| ligand 3 | (SEQ ID NO: 9) |
| | A: 5′-TCCTGTCAGTTGGTGCTCC -3′ |
| | (SEQ ID NO: 10) |

Immunofluorescence:

PPDCs were fixed with cold 4% (w/v) paraformaldehyde (Sigma-Aldrich, St. Louis, Mo.) for 10 minutes at room temperature. One isolate each of umbilicus- and placenta-derived cells at passage 0 (PO) (directly after isolation) and passage 11 (P 11) (two isolates of placenta-derived, two isolates of umbilicus-derived cells) and fibroblasts (P 11) were used. Immunocytochemistry was performed using antibodies directed against the following epitopes: vimentin (1:500, Sigma, St. Louis, Mo.), desmin (1:150; Sigma—raised against rabbit; or 1:300; Chemicon, Temecula, Calif.—raised against mouse,), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested on passage 11 postpartum cells: anti-human GRO alpha—PE (1:100; Becton Dickinson, Franklin Lakes, N.J.), anti-human GCP-2 (1:100; Santa Cruz Biotech, Santa Cruz, Calif.), anti-human oxidized LDL receptor 1 (ox-LDL R1; 1:100; Santa Cruz Biotech), and anti-human NOGA-A (1:100; Santa Cruz, Biotech).

Cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma, St. Louis, Mo.) for 30 minutes to access intracellular antigens. Where the epitope of interest was located on the cell surface (CD34, ox-LDL R1), Triton X-100 was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout. Primary antibodies, diluted in blocking solution, were then applied to the cultures for a period of 1 hour at room temperature. The primary antibody solutions were removed and the cultures were washed with PBS prior to application of secondary antibody solutions (1 hour at room temperature) containing block along with goat anti-mouse IgG—Texas Red (1:250; Molecular Probes, Eugene, Oreg.) and/or goat anti-rabbit IgG—Alexa 488 (1:250; Molecular Probes) or donkey anti-goat IgG—FITC (1:150, Santa Cruz Biotech). Cultures were then washed and 10 micromolar DAPI (Molecular Probes) applied for 10 minutes to visualize cell nuclei.

Following immunostaining, fluorescence was visualized using an appropriate fluorescence filter on an Olympus® inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro® software (Media Cybernetics, Carlsbad, Calif.). For triple-stained samples, each image was taken using only one emission filter at a time. Layered montages were then prepared using Adobe Photoshop® software (Adobe, San Jose, Calif.).

Preparation of Cells for FACS Analysis:

Adherent cells in flasks were washed in phosphate buffered saline (PBS) (Gibco, Carlsbad, Calif.) and detached with Trypsin/EDTA (Gibco, Carlsbad, Calif.). Cells were harvested, centrifuged, and re-suspended 3% (v/v) FBS in PBS at a cell concentration of 1×10 7 per milliliter. One hundred microliter aliquots were delivered to conical tubes. Cells stained for intracellular antigens were permeabilized with Perm/Wash buffer (BD Pharmingen, San Diego, Calif.). Antibody was added to aliquots as per manufactures specifications and the cells were incubated for in the dark for 30 minutes at 4° C. After incubation, cells were washed with PBS and centrifuged to remove excess antibody. Cells requiring a secondary antibody were resuspended in 100 microliters of 3% FBS. Secondary antibody was added as per manufactures specification and the cells were incubated in the dark for 30 minutes at 4° C. After incubation, cells were washed with PBS and centrifuged to remove excess secondary antibody. Washed cells were resuspended in 0.5 milliliters PBS and analyzed by flow cytometry. The following antibodies were used: oxidized LDL receptor 1 (sc-5813; Santa Cruz, Biotech), GROa (555042; BD Pharmingen, Bedford, Mass.), Mouse IgG1 kappa, (P-4685 and M-5284; Sigma), Donkey against Goat IgG (sc-3743; Santa Cruz, Biotech.). Flow cytometry analysis was performed with FACScalibur™ (Becton Dickinson San Jose, Calif.).

Results

Results of real-time PCR for selected “signature” genes performed on cDNA from cells derived from human placentae, adult and neonatal fibroblasts and Mesenchymal Stem Cells (MSCs) indicate that both oxidized LDL receptor and rennin were expressed at higher level in the placenta-derived cells as compared to other cells. The data obtained from real-time PCR were analyzed by the AACT method and expressed on a logarithmic scale. Levels of reticulon and oxidized LDL receptor expression were higher in umbilicus-derived cells as compared to other cells. No significant difference in the expression levels of CXC ligand 3 and GCP-2 were found between postpartum-derived cells and controls. The results of real-time PCR were confirmed by conventional PCR. Sequencing of PCR products further validated these observations. No significant difference in the expression level of CXC ligand 3 was found between postpartum-derived cells and controls using conventional PCR CXC ligand 3 primers listed above in Table 7-1.

The production of the cytokine, IL-8 in postpartum was elevated in both Growth Medium-cultured and serum-starved postpartum-derived cells. All real-time PCR data was validated with conventional PCR and by sequencing PCR products.

When supernatants of cells grown in serum-free medium were examined for the presence of IL-8, the highest amounts were detected in media derived from umbilical cells and some isolates of placenta cells (Table 7-2). No IL-8 was detected in medium derived from human dermal fibroblasts.

TABLE 7-2
IL-8 protein expression measured by ELISA

| Cell type | IL-8 |
|---|---|
| Human fibroblasts | ND |
| Placenta Isolate 1 | ND |
| UMBC Isolate 1 | 2058.42 ± 144.67 |
| Placenta Isolate 2 | ND |
| UMBC Isolate 2 | 2368.86 ± 22.73 |
| Placenta Isolate3 (normal O₂) | 17.27 ± 8.63 |
| Placenta Isolate 3 (lowO₂, W/O BME) | 264.92 ± 9.88 |

Results of the ELISA assay for interleukin-8 (IL-8) performed on placenta-and umbilical cord-derived cells as well as human skin fibroblasts.
Values are presented here are picogram/million cells, n = 2, sem.
ND: Not Detected

Placenta-derived cells were also examined for the production of oxidized LDL receptor, GCP-2 and GROalpha by FACS analysis. Cells tested positive for GCP-2. Oxidized LDL receptor and GRO were not detected by this method.

Placenta-derived cells were also tested for the production of selected proteins by immunocytochemical analysis. Immediately after isolation (passage 0), cells derived from the human placenta were fixed with 4% paraformaldehyde and exposed to antibodies for six proteins: von Willebrand Factor, CD34, cytokeratin 18, desmin, alpha-smooth muscle actin, and vimentin. Cells stained positive for both alpha-smooth muscle actin and vimentin. This pattern was preserved through passage 11. Only a few cells (<5%) at passage 0 stained positive for cytokeratin 18.

Cells derived from the human umbilical cord at passage 0 were probed for the production of selected proteins by immunocytochemical analysis. Immediately after isolation (passage 0), cells were fixed with 4% paraformaldehyde and exposed to antibodies for six proteins: von Willebrand Factor, CD34, cytokeratin 18, desmin, alpha-smooth muscle actin, and vimentin. Umbilicus-derived cells were positive for alpha-smooth muscle actin and vimentin, with the staining pattern consistent through passage 11.

Summary:

Concordance between gene expression levels measured by microarray and PCR (both real-time and conventional) has been established for four genes: oxidized LDL receptor 1, rennin, reticulon, and IL-8. The expression of these genes was differentially regulated at the mRNA level in PPDCs, with IL-8 also differentially regulated at the protein level. The presence of oxidized LDL receptor was not detected at the protein level by FACS analysis in cells derived from the placenta. Differential expression of GCP-2 and CXC ligand 3 was not confirmed at the mRNA level, however GCP-2 was detected at the protein level by FACS analysis in the placenta-derived cells. Although this result is not reflected by data originally obtained from the micro array experiment, this may be due to a difference in the sensitivity of the methodologies.

Immediately after isolation (passage 0), cells derived from the human placenta stained positive for both alpha-smooth muscle actin and vimentin. This pattern was also observed in cells at passage 11. Vimentin and alpha-smooth muscle actin expression may be preserved in cells with passaging, in the Growth Medium and under the conditions utilized in these procedures. Cells derived from the human umbilical cord at passage 0 were probed for the expression of alpha-smooth muscle actin and vimentin, and were positive for both. The staining pattern was preserved through passage 11.

### Example 8

### Telomerase Expression in Umbilical Tissue-Derived Cells

Telomerase functions to synthesize telomere repeats that serve to protect the integrity of chromosomes and to prolong the replicative life span of cells (Liu, K, et al., PNAS, 1999; 96:5147-5152). Telomerase consists of two components, telomerase RNA template (hTER) and telomerase reverse transcriptase (hTERT). Regulation of telomerase is determined by transcription of hTERT but not hTER. Real-time polymerase chain reaction (PCR) for hTERT mRNA thus is an accepted method for determining telomerase activity of cells.

Cell Isolation.

Real-time PCR experiments were performed to determine telomerase production of human umbilical cord tissue-derived cells. Human umbilical cord tissue-derived cells were prepared in accordance the examples set forth above. Generally, umbilical cords obtained from National Disease Research Interchange (Philadelphia, Pa.) following a normal delivery were washed to remove blood and debris and mechanically dissociated. The tissue was then incubated with digestion enzymes including collagenase, dispase and hyaluronidase in culture medium at 37° C. Human umbilical cord tissue-derived cells were cultured according to the methods set forth in the examples above. Mesenchymal stem cells and normal dermal skin fibroblasts (cc-2509 lot #9F0844) were obtained from Cambrex, Walkersville, Md. A pluripotent human testicular embryonal carcinoma (teratoma) cell line nTera-2 cells (NTERA-2 cl.D1), (see, Plaia et al., Stem Cells, 2006; 24(3):531-546) was purchased from ATCC (Manassas, Va.) and was cultured according to the methods set forth above.

Total RNA Isolation.

RNA was extracted from the cells using RNeasy® kit (Qiagen, Valencia, Ca.). RNA was eluted with 50 microliters DEPC-treated water and stored at −80° C. RNA was reverse transcribed using random hexamers with the TaqMan® reverse transcription reagents (Applied Biosystems, Foster City, Ca.) at 25° C. for 10 minutes, 37° C. for 60 minutes and 95° C. for 10 minutes. Samples were stored at −20° C.

Real-Time PCR.

PCR was performed on cDNA samples using the Applied Biosystems Assays-On-Demand™ (also known as TaqMan® Gene Expression Assays) according to the manufacturer's specifications (Applied Biosystems). This commercial kit is widely used to assay for telomerase in human cells. Briefly, hTert (human telomerase gene) (Hs00162669) and human GAPDH (an internal control) were mixed with cDNA and TaqMan® Universal PCR master mix using a 7000 sequence detection system with ABI prism 7000 SDS software (Applied Biosystems). Thermal cycle conditions were initially 50° C. for 2 minutes and 95° C. for 10 minutes followed by 40 cycles of 95° C. for 15 seconds and 60° C. for 1 minute. PCR data was analyzed according to the manufacturer's specifications.

Human umbilical cord tissue-derived cells (ATCC Accession No. PTA-6067), fibroblasts, and mesenchymal stem cells were assayed for hTert and 18S RNA. As shown in Table 8-1, hTert, and hence telomerase, was not detected in human umbilical cord tissue-derived cells.

TABLE 8-1

| | hTert | 18S RNA |
|---|---|---|
| Umbilical cells (022803) | ND | + |
| Fibroblasts | ND | + |

ND-not detected; + signal detected

Human umbilical cord tissue-derived cells (isolate 022803, ATCC Accession No. PTA-6067) and nTera-2 cells were assayed and the results showed no expression of the telomerase in two lots of human umbilical cord tissue-derived cells while the teratoma cell line revealed high level of expression (Table 8-2).

TABLE 8-2

| Cell | hTert | | GAPDH | | hTert |
|---|---|---|---|---|---|
| type | Exp. 1 | Exp. 2 | Exp. 1 | Exp. 2 | norm |
| nTera2 | 25.85 | 27.31 | 16.41 | 16.31 | 0.61 |
| 022803 | — | — | 22.97 | 22.79 | — |

Therefore, it can be concluded that the human umbilical tissue-derived cells of the present invention do not express telomerase.

Various patents and other publications are referred to throughout the specification. Each of these publications is incorporated by reference herein, in its entirety.

Although the various aspects of the invention have been illustrated above by reference to examples and preferred embodiments, it will be appreciated that the scope of the invention is defined not by the foregoing description but by the following claims properly construed under principles of patent law.

In describing the present invention and its various embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

What is claimed is:

1. A method of inhibiting or reducing retinal neovascularization in retinopathy comprising administering a homogeneous population of human umbilical cord tissue-derived cells to the eye of a subject, wherein the cell population is isolated from human umbilical cord tissue substantially free of blood, is capable of expansion in culture, expresses CD13, CD90 and HLA-ABC, and does not express CD31, CD34, CD45 and CD117.

2. The method of claim 1 wherein the cell population further has the following characteristics:
a) potential for 40 population doublings in culture;
b) expresses CD10, CD44 and CD73;
c) does not express CD141; and
d) lack expression of hTERT or telomerase.

3. The method of claim 1, wherein the cell population has increased expression of genes encoding interleukin 8 and reticulon 1 relative to a human cell that is a fibroblast, a mesenchymal stem cell, or an iliac crest bone marrow cell.

4. A method of producing a conditioned media comprising human VEGFR1, wherein the conditioned media is prepared from a homogeneous population of human umbilical cord tissue-derived cells, wherein the cell population is isolated from human umbilical cord tissue substantially free of blood.

5. The method of claim 4, wherein the cell population further has the following characteristics:
a) potential for 40 population doublings in culture;
b) expresses CD10, CD44 and CD73;
c) does not express CD141; and
d) lack expression of hTERT or telomerase.

6. The method of claim 4, wherein the cell population has increased expression of genes encoding interleukin 8 and reticulon 1 relative to a human cell that is a fibroblast, a mesenchymal stem cell, or an iliac crest bone marrow cell.

7. A method of inhibiting or reducing retinal neovascularization in retinopathy comprising administering the conditioned medium produced in claim 4 to the eye of a subject with the retinopathy.

8. A composition for use in reducing neoovascularization comprising VEGFR1 and a homogeneous population of human umbilical cord tissue-derived cells, wherein the cell population is isolated from human umbilical cord tissue substantially free of blood, is capable of expansion in culture, expresses CD13, CD90 and HLA-ABC, and does not express CD31, CD34, CD45 and CD117.

9. The composition of claim 8, wherein the cell population further has the following characteristics:
a) potential for 40 population doublings in culture;
b) expresses CD10, CD44 and CD73;
c) does not express CD141; and
d) lack expression of hTERT or telomerase.

10. The composition of claim 8, wherein the cell population has increased expression of genes encoding interleukin 8 and reticulon 1 relative to a human cell that is a fibroblast, a mesenchymal stem cell, or an iliac crest bone marrow cell.
